(19) United States
(12) Patent Application Publication
(10) Pub. No.: US 2022/0241200 A1
(43) Pub. Date: Aug. 4, 2022

(54) COMPOSITIONS AND METHODS FOR SUSTAINED TREATMENT OF PAIN

(71) Applicant: InSitu Biologics, Inc., Woodbury, MN (US)

(72) Inventor: William J. Taylor, Woodbury, MN (US)

(21) Appl. No.: 17/589,639

(22) Filed: Jan. 31, 2022

Related U.S. Application Data
(60) Provisional application No. 63/143,542, filed on Jan. 29, 2021.
(60) Provisional application No. 63/232,027, filed on Aug. 11, 2021.

(51) Int. Cl. A61K 9/107; A61K 31/445; A61K 47/12

(52) U.S. Cl. CPC A61K 9/1075; A61K 47/12; A61K 31/445

(57) ABSTRACT

Disclosed herein is a composition for treating post-surgical pain comprising: an aqueous carrier; and a lipid phase comprising an anesthetic agent, the lipid phase dispersed within the aqueous carrier. In certain aspects, the aqueous carrier is hydrogel comprised of tyramine substituted hyaluronic acid. In certain embodiments, the hydrogel is formed through di-tyramine crosslinking. In certain embodiments, the degree of tyramine substitution of hyaluronic acid hydroxyl groups is about 0.5% to about 3%. In further aspects, the lipid phase is comprised of a plurality of lipid microparticles. According to certain embodiments, a salt form of the anesthetic unbound by the plurality of lipid microparticles is dissolved in the aqueous carrier.

### CROSS-REFERENCE TO RELATED APPLICATION(S)

This application claims the benefit under 35 U.S.C. § 119(e) to U.S. Provisional Application 63/143,542, filed Jan. 29, 2021, and entitled “Compositions and Methods for Sustained Treatment of Pain,” which is hereby incorporated herein by reference in its entirety for all purposes.

### BACKGROUND

Management of post-operative pain is a significant challenge and use of opiate medications carries the risk of side effects, tolerance, and long term dependence. There is a need in the art for compositions and methods that are effective in providing sustained post-operative pain relief without the use of opiate-based medications.

### BRIEF SUMMARY

Disclosed herein is a composition for treating post-surgical pain comprising: an aqueous carrier; and a lipid phase comprising an anesthetic agent, the lipid phase dispersed within the aqueous carrier. In certain aspects, the aqueous carrier is hydrogel comprised of tyramine substituted hyaluronic acid. In certain embodiments, the hydrogel is formed through di-tyramine crosslinking. In certain embodiments, the degree of tyramine substitution of hyaluronic acid hydroxyl groups is about 0.5% to about 3%.

In certain aspects, the lipid phase is emulsified within the aqueous carrier.

In further aspects, the lipid phase is comprised of a plurality of lipid microparticles. According to certain embodiments, a salt form of the anesthetic unbound by the plurality of lipid microparticles is dissolved in the aqueous carrier. In exemplary implementations, the volumetric ratio between the aqueous carrier and the lipid microparticles is from about 70-80 aqueous carrier: 30-20 lipid microparticles.

In certain aspects, the lipid microparticles comprise one or more fatty acids having an even number of carbons. In further aspects, the lipid microparticles comprise one or more fatty acids having an odd number of carbons. According to certain embodiments, the one or more fatty acids are chosen from: stearic acid, oleic acid, myristic acid, caprylic acid, capric acid, lauric acid, palmitic acid, arachidic acid, lignoceric acid, cerotic acid, and mixtures of the forgoing. In certain implementations, the melting point of the lipid microparticle is above 37° C. In further implementations, the melting point of the lipid microparticle is below 37° C.

According to certain embodiments, the one or more fatty acids comprise a mixture of steric acid and oleic acid and wherein the ratio of steric acid to oleic acid is about 90:10. In further embodiments, the lipid microparticles comprise about 12% myristic acid, about 32% palmitic acid, about 10% stearic acid, and about 10% oleic acid. In yet further embodiments, the lipid microparticles comprise a mixture of lauric acid and caprylic acid, caproic acid, and/or oleic acid. In still further embodiments, the lipid microparticle comprises a paraffin, a triglyceride, and/or a wax. In exemplary implementations of these embodiments, the lipid microparticles comprise a mixture of carnauba wax and caprylic acid, caproic acid, and/or oleic acid.

According to certain embodiments, the plurality of lipid microparticles comprises a first plurality of lipid microparticles and a second plurality of lipid microparticles and wherein the first plurality of lipid microparticles is solid at about 37° C. and the second plurality of lipid microparticles is liquid at 37° C.

In certain embodiments, lipid microparticle is not a liposome.

In certain embodiments, the lipid microparticle ranges in size from about 1 μm to about 20 μm. In exemplary implementations, the lipid microparticle ranges in size from about 4 μm to about 8 μm.

According to certain embodiments, the anesthetic agent comprises ropivacaine. In certain implementations, ropivacaine is present in the lipid microparticles in an amount of from about 1 to about 25% by weight.

Further disclosed herein is a composition for treating post-surgical pain comprising an aqueous carrier; a first lipid phase comprising a plurality of lipid microparticles comprising an anesthetic agent and dispersed within the aqueous carrier; and a second lipid phase comprising an anesthetic agent dissolved in one or more lipids and emulsified into to the aqueous phase. In certain implementations, a salt form of the anesthetic agent, not present in the first lipid phase or the second lipid phase, is dissolved in the aqueous carrier.

According to certain embodiments, the one or more lipids of the second lipid phase is one or more fatty acids and the second lipid phase is emulsified into the aqueous phase. In exemplary implementations, the one or more fatty acids of the second lipid phase are a mixture of stearic acid and oleic acid.

According to certain embodiments, the volumetric ratio of the first lipid phase and the second lipid phase is about 66:34.

Further disclosed herein is a method of treating post-operative pain in a subject in need thereof comprising administering to the subject and effective amount of a composition comprising: an immiscible carrier phase and a plurality of lipid microparticles dispersed within the immiscible carrier phase comprising an anesthetic agent. In certain embodiments, the immiscible carrier phase is a hydrogel, a viscous liquid, a stable emulsion, or a cream. In further embodiments, the immiscible carrier phase is a hydrogel. In exemplary implementations, the hydrogel is comprised of tyramine substituted hyaluronic acid and wherein the anesthetic agent is ropivacaine. According to certain embodiments, the composition is administered to the subject and wherein the composition provides pain relief for about 72 hours.

According to certain embodiments, the composition is delivered near a never or nerve bundle of a subject and wherein the nerve or nerve bundle innervates the surgical incision area of the subject.

Further disclosed herein is a composition for sustained release of an active pharmaceutical ingredient (API) comprising: a hydrogel; and a plurality of lipid microparticles dispersed within the hydrogel comprising the API. In certain embodiments the API is a chemotherapeutic composition. In further embodiments, the API is a motion sickness drug. In exemplary implementations, the motion sickness drug is meclizine or dimenhydrinate. In further implementations, the API is selected from NSAIDS, steroids, biologics such as antibodies, hormones.

While multiple embodiments are disclosed, still other embodiments of the disclosure will become apparent to those skilled in the art from the following detailed description, which shows and describes illustrative embodiments of the disclosed apparatus, systems, and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows representative images of the disclosed hydrogel composition at varying ionic concentrations, according to certain embodiments.

FIG. 2 shows representative images of the disclosed hydrogel, according to certain embodiments.

FIG. 3 shows representative images of the disclosed hydrogel, according to certain embodiments.

FIG. 4 shows representative images of the disclosed hydrogel, according to certain embodiments.

FIG. 5 shows data from animal studies indicating sustained release of an API, according to certain embodiments.

FIG. 6 shows data from a study of a 200 mg equivalent Ropivacaine HCl in aqueous carrier phase with 3 different ropivacaine base loadings in the lipid drug reservoir phase compared to Naropin (ropivaine HCl injection) and Exparel (Bupivacaine sustained release) controls.

FIG. 7 shows comparisons of formulations that contain an upfront burst of ropivacaine HCl compared to formulations that do not contain ropivacaine HCl in the aqueous carrier phase.

FIG. 8 shows a comparison of Variable Aqueous phase Ropivacaine HCl concentration.

FIG. 9 shows a comparison of formulations based on lipid phase volume percent.

FIG. 10 shows comparisons of low loaded drug reservoirs based on lipid phase volume percent.

FIG. 11 shows a comparison of midlevel ropivacaine concentration in the lipid phase reservoir.

FIG. 12 shows a comparison of midlevel ropivacaine concentration in the lipid phase reservoir.

FIG. 13 shows a comparison of solid lipid phase drug reservoirs to emulsion phase drug reservoir.

FIG. 14 shows a comparison between a solid phase drug reservoir, an emulsion phase drug reservoir and combination drug reservoir formulations.

FIG. 15 shows a comparison of drug loading levels in an emulsion phase drug reservoir formulation.

FIG. 16 shows a comparison of various ratios of solid phase to emulsion phase drug reservoirs on elution rate.

FIG. 17 shows a comparison of 20 mL dose and 30 mL dose of sample 9LL.

FIG. 18 shows a comparison of 20 mL dose and 30 mL dose of Laurie acid based formulations.

FIG. 19 shows a comparison of 20 mL dose and 30 mL dose of Carnauba Wax formulations.

FIG. 20 shows a comparison of high concentration to low concentration formulations.

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “subject” refers to the target of administration, e.g. a subject. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the terms “treat,” and “prevent” as well as words stemming therefrom, do not necessarily imply 100% or complete treatment or prevention. Rather, there are varying degrees of treatment or prevention of which one of ordinary skill in the art recognizes as having a potential benefit or therapeutic effect. In this respect, the methods of the present invention can provide any amount of any level of treatment or prevention of a disease or medical condition in a mammal. Furthermore, the treatment or prevention provided by the method can include treatment or prevention of one or more conditions or symptoms of the disease or medical condition. For example, with regard to methods of treating pain, the method in some embodiments, achieves a diminution in or elimination of pain in a subject. Also, for purposes herein, “prevention” can encompass delaying the onset of the disease, or a symptom or condition thereof. The term “treating” includes prophylaxis of the specific disorder or condition, or alleviation of the symptoms associated with a specific disorder or condition and/or preventing or eliminating said symptoms. For example, as used herein the term “post-operative pain” refers in general to producing a diminution or alleviation of pain associated with recovering from a surgical procedure.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles” would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, the term “active pharmaceutical ingredient,” or API, refers to a molecular entity adapted for treatment of a malcondition in a subject in need thereof.

The term “anesthetic agent” or “local anesthetic agent” (used unteachably herein) refers to an agent that causes loss of sensation in a human or other mammal with or without the loss of consciousness. More particularly, the term “local anesthetic” refers to an anesthetic agent that induces local anesthesia by reversibly inhibiting peripheral nerve excitation and/or conduction. Local anesthetics suitable for use in the present invention include, but are not limited to, ester-based anesthetics, amide-based anesthetics, ester analogs of amide-based anesthetics, and ester analogs of other anesthetics. Ester-based anesthetics include, but are not limited to, cocaine, procaine, 2-chloroprocaine, tetracaine, benzocaine, amethocaine, chlorocaine, butamben, dibucaine, and the like. Amide-based anesthetics include, but are not limited to, lidocaine, prilocaine, mepivacaine, ropivacaine, etidocaine, levobupivacaine, bupivacaine, and the like. Other anesthetics suitable for use in the present invention include, but are not limited to, ester analogs of aconitine, dyclonine, ketamine, pramoxine, safrole, and salicyl alcohol. Such ester analogs can contain an ester group anywhere within the structure.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations, taking into account the bioavailability of the particular active agent, is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, which is hereby incorporated by reference in its entirety, and the references cited therein.

As used herein, “drug reservoir” means a phase into which an API is dissolved that is dissolved distinct from the carrier phase.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Disclosed herein are compositions for providing controlled and/or sustained release of an API (particularly, hydrophobic APIs) in the body of a subject. In certain aspects, disclosed herein is composition for treating post-surgical pain comprising a hydrogel and a plurality of lipid microparticles dispersed within the hydrogel comprising an anesthetic agent.

According to certain implementations, the disclosed composition comprises a carrier phase and a drug reservoir phase that contains an API (e.g., an anesthetic agent) that is released to a biological system over a targeted treatment duration. In this context the primary function of the carrier phase is to disperse the drug reservoir particles (drug carrying component) to create a stable homogenous mass and allow the use of delivery devices, such as a syringe, to draw up a dose from a container and deliver it to a target tissue, i.e. parenteral injection, intravascular injection, wound instillation, wound packing or mass formation or coating on a tissue surface. In certain embodiments, the drug reservoir is a separate physical phase, a collection of particles, that are contained within the carrier phase but not indistinguishable from the carrier phase. The reservoir phase contains the active pharmaceutical agent dissolved in the reservoir material and may be in an unsaturated, saturated, super saturated, or saturated with pure pharmaceutical phase material (crystals for small molecules) state. In some forms the carrier may also contain the contain the API in a different form than the reservoir, such as an API salt in an aqueous carrier and the base form API in a lipid reservoir. The system is not set to be only aqueous/hydrophobic, but can be opposite, or separate physical phase (polymer).

In certain embodiments, the carrier phase is a hydrogel. The term “hydrogel” as used herein refers to a three-dimensional, hydrophilic or amphiphilic polymeric network capable of taking up large quantities of water. The networks are composed of homopolymers or copolymers (referred to at times herein as a polymer backbone) and are insoluble due to the presence of covalent chemical or physical (ionic, hydrophobic interactions, entanglements) crosslinks. The crosslinks provide the network structure and physical integrity. Hydrogels exhibit a thermodynamic compatibility with water that allow them to swell in aqueous media.

In certain implementations, the hydrogel is comprised of tyramine substituted hyaluronic acid (THA) which is cross linked through di-tyramine linkages. Preparation of THA is described U.S. Pat. No. 6,982,298, which is incorporated herein by reference in its entirety. The degree of tyramine substitution has a significant impact on the properties of the resulting hydrogel. Throughout the instant disclosure, degree of tyramine substitution refers to the percentage of all HA carboxyl groups that have been substituted by tyramine. For example, in a 2% substituted THA, 2% of all HA carboxyl groups have been substituted by tyramine. The percent tyramine substitution within each THA preparation is calculated by measuring: 1) the concentration of tyramine present in the preparation, which is quantitated spectrophotometrically based on the unique UV-absorbance properties of tyramine at 275 nm; and 2) the concentration of total carboxyl groups in the HA preparation, which is quantitated spectrophotometrically by a standard hexuronic acid assay.

As described further below, hydrogel can be tuned to possess a specific osmolality, physical property, API elution rate or tissue response by adjusting the concentration of the tyramine substituted polymer backbone, the degree of substitution of the tyramine on the polymer backbone, the molecular weight of polymer backbone, the hydrophilicity of the polymer backbone, the type of polymer backbone and concentration of target molecules, salts, buffers or drug depot (reservoir) particles contained within the hydrogel.

The hydrogel physical properties can be adjusted by changing the concentration of tyramine substituted polymer backbone. In certain embodiments, liquid-like hydrogels are created by keeping the tyramine substituted polymer backbone less than 0.35% of the aqueous carrier phase for a 1.5% substituted gel. Liquid-like hydrogels are more appropriate for intravascular injection, intrathecal injection or other tissue sites that cannot tolerate occlusion or blocking vessels or tissue structures. Dense hydrogel particles can be formed by increasing tyramine substitution on the polymer backbone. 5% or higher degrees of substitution will form solid like hydrogel particles at low concentrations and very dense particles at 7% or higher concentrations. Dense particles are more appropriate for instillation into wound sites. In certain implementations, dense hydrogel particles are used deliver biological molecules and polar APIs. In contrast, in implementations where the API is hydrophobic, lipid microparticles are suitable.

The hydrogel physical property can also be adjusted by changing the type of polymer backbone. For example, collagen can be used as a polymer backbone, and it is much less hydrophilic than a saccharide-based polymer backbone. The collagen gels do not swell in the same way that polysaccharide gels and have much lower molecular weights and concentrations. It can be envisioned that the polymer backbone can be changed to take advantage of a single polymers physical & chemical characteristics, or several species can be combined in a copolymer or block copolymer in a way that will change the gel physical and chemical properties, the way in which the body interacts with the gel. Some polymers will have a higher affinity rate for an API and API elution rates will be impacted if a polymer or section of polymer has been chosen that has a higher binding affinity for the API. It is also envisioned that by using polymer/API combinations in which binding affinity of the API to the backbone polymer is pH or temperature dependent, the gel formulation can be adjusted to maximize binding at T=0 and then releasing more API as the pH and temperature approaches physiological conditions after exposure to target tissues. In further implementations, API diffusion rate is affected by changing the melting point of the lipid microparticles (described further below) as enhanced diffusion can be reached as the liquid-liquid interface (achieved upon melting of the lipid microparticle) diffusion flux is higher than solid to liquid interface.

The hydrogel osmolality can also be tuned by the degree of tyramine substitution, and concentration. Concentrated highly substituted hydrogels by themselves will expel water or undergo syneresis, but by increasing the concentration of the polymer backbone in the example of hyaluronic acid, or adding salts, buffers and/or API materials to the formulation the gel can be made to be osmotically neutral or swell slightly. For example, a 5.5% substituted gel can be created that will swell if the backbone polymer concentration is set to 1.5%. It is envisioned that a gel can be created to swell even more as the osmolality of the gel is increased by adding buffers, salts and API ingredients. In certain aspects, the hydrogel is comprised of tyramine substituted hyaluronic acid. According to certain implementations, the hydrogel is formed through di-tyramine crosslinking.

The advantage of controlling backbone polymer concentration and degree of substitution can also be used to elicit a biological response. Using a gel with 1.5% substitution and 0.5% concentration will absorb fluids from the tissues surrounding the hydrogel implant. The capillary beds will constrict and in some cases such as a traumatic wound site will decrease or stop bleeding from a wounded surface. The reduction of blood flow in tissues near the implant will also slow removal of an eluting API from the implant site. Tissues which may be harmed from reduced perfusion such as cartilage or joint spaces can have a hydrogel tuned to be osmotically neutral to prevent negative impacts due to reduced perfusion. In clinical applications where it is desirable to stop bleeding from a highly vascular bleeding surface such as the liver, a very concentrated hydrogel that appears to be dry or almost dry will absorb sera and exudate very quickly and dehydrate the wound site. In the case where procoagulants such as fibrin, tranexamic acid, aminocaproic acid or fibrin, etc. are employed, the hydrogel can promote coagulation at the wound site via two pathways, capillary bed constriction and blood coagulation.

Hydrogel density can be used to control rate of elution of an API from the gel to the target tissue. A 1% hydrogel will elute API for >72 hrs, but a 10% gel will extend elution time to over 100 hrs. Depending on API or biologic material size and affinity for the hydrogel components, the elution rate can be tuned to a desired elution rate that will allow the hydrogel to act as a drug reservoir for several days.

In certain aspects, the degree of tyramine substitution of hyaluronic acid hydroxyl groups ranges from about 0.25% to about 8%. In further aspects, the degree of tyramine substitution of hyaluronic acid hydroxyl groups is about 0.5% to about 3%.

In still further aspects, the tyramine substituted hyaluronic acid is present in the aqueous phase at from about 0.1% to about 4%.

In certain implementations, the tyramine substituted hyaluronic acid is present in the aqueous phase from about 0.1 to about 1%. In further implementations, the tyramine substituted hyaluronic acid is present in the aqueous phase at about 0.25%.

### Lipid Microparticles

According to certain embodiments, lipid microparticles of the disclosed composition are comprised of one or more fatty acids. In certain implementations, the one or more fatty acids have an even number of carbons. In certain implementations, the fatty acids are chosen from: stearic acid, oleic acid, myristic acid, caprylic acid, capric acid, lauric acid, palmitic acid, arachidic acid, lignoceric acid, cerotic acid, and mixtures of the forgoing.

In certain exemplary implementation, where the fatty acid microparticles are comprised of mixtures of fatty acids, the fatty acids are present at specific ratios. For example, in certain implementations, the mixture of fatty acids comprises a 90:10 ratio of steric to oleic acid.

Fatty acids of various carbon lengths are common throughout the living world and are utilized by animals as part of the cell membrane, as energy storage and for thermal regulation. Fatty acids are comprised of carboxylic acid attached to an aliphatic carbon chain. In general, they are insoluble in water but as the carbon chain length shortens, their acidity increases. Fatty acids can be saturated or contain no carbon-carbon double bonds. Or they may be unsaturated, containing one or more carbon-carbon double bonds in the aliphatic carbon chain. Mammalian organisms can process and create fatty acids with even numbered carbon chains. Odd numbered fatty acids are produced by some bacteria and are found in the milk of ruminants, but in most cases, they are even numbered due to the metabolic process that adds two carbons at a time to the chain. Table 1 lists fatty acids typically found in plant and animals. The lipid number lists the number of carbons in the aliphatic chain followed by the number of double bonds. In some listings, the location of the double bond is included with the lipid number. In most cases the fatty acids are usually part of a triglyceride molecule that may contain up to three fatty acids of the same or differing carbon lengths.

In certain implementations, even numbered carbon fatty acids are selected. Mixtures of fatty acids can be made to adjust the melting point of the microparticles. In certain implementations, a mixture of 90% stearic acid with 10% oleic acid is used. This creates a microparticle that melts at 95° F. A similar melting point is achieved by mixing 12% myristic acid 32% palmitic acid, 10% stearic acid, and 10% oleic acid. According to further embodiments, the fatty acid microparticle is formed from a mixture of lauric acid, caprylic acid, and caproic acid. The key factors in choosing a microparticle formulation are melting point and API solubility in main component fatty acid. The melting point is important in that particles close to physiological body temperature will be a liquid or soft semi-solid which will increase diffusion rate across a liquid-liquid interface. This may be desirable or not desirable depending on the specific application. In certain embodiments, a combination of low melting point and high melting microparticles (e.g. below and above 37 C) are combined. API solubility will change due to fatty acid chain length and microparticle formulation and it may be desirable to adjust API concentration and affinity for the main microparticle fatty acid component. In some formulations increasing molecular weight and chain length of the fatty acid will change solubility of a partially polar API counterintuitively. In certain embodiments, the concentration of anesthetic agent within a fatty acid microparticle is from about 1-25% by weight.

According to certain alternative embodiments, odd numbered fatty acids are used as an alternative fatty acid in the formulations. Monounsaturated fatty acids such as oleic acid may be used as well alone or in combination with other fatty acids. In certain implementations, poly unsaturated fatty acids can be used, but are not preferable as they oxidize easily and depending on the formulation may polymerize. Monounsaturated fatty acids that are in a cis configuration (most plant sourced) are preferable.

According to certain alternative embodiments, the lipid microparticles comprise one or more triglyceride or a mixture of triglycerides the lipid microparticles comprise one or more triglyceride or a mixture of triglycerides. In further alternative embodiments, the lipid microparticle comprises a paraffin and/or a wax.

TABLE 1
List of Fatty Acids and Corresponding Lipid Numbers.

| | | | Lipid |
|---|---|---|---|
| Common Name | Chemical Name | Structural Formula | Numbers |
| Propionic acid | Propanoic acid | CH₃CH₂COOH | C3:0 |
| Butyric acid | Butanoic acid | CH₃(CH₂)₂COOH | C4:0 |
| Valeric acid | Pentanoic acid | CH₃(CH₂)₃COOH | C5:0 |
| Caproic acid | Hexanoic acid | CH₃(CH₂)₄COOH | C6:0 |
| Enanthic acid | Heptanoic acid | CH₃(CH₂)₅COOH | C7:0 |
| Caprylic acid | Octanoic acid | CH₃(CH₂)₆COOH | C8:0 |
| Pelargonic acid | Nonanoic acid | CH₃(CH₂)₇COOH | C9:0 |
| Capric acid | Decanoic acid | CH₃(CH₂)₈COOH | C10:0 |
| Undecylic acid | Undecanoic acid | CH₃(CH₂)₉COOH | C11:0 |
| Lauric acid | Dodecanoic acid | CH₃(CH₂)₁₀COOH | C12:0 |
| Tridecylic acid | Tridecanoic acid | CH₃(CH₂)₁₁COOH | C13:0 |
| Myristic acid | Tetradecanoic acid | CH₃(CH₂)₁₂COOH | C14:0 |
| Pentadecylic acid | Pentadecanoic acid | CH₃(CH₂)₁₃COOH | C15:0 |
| Palmitic acid | Hexadecanoic acid | CH₃(CH₂)₁₄COOH | C16:0 |
| Margaric acid | Heptadecanoic acid | CH₃(CH₂)₁₅COOH | C17:0 |
| Stearic acid | Octadecanoic acid | CH₃(CH₂)₁₆COOH | C18:0 |
| Nonadecylic acid | Nonadecanoic acid | CH₃(CH₂)₁₇COOH | C19:0 |
| Arachidic acid | Eicosanoic acid | CH₃(CH₂)₁₈COOH | C20:0 |
| Heneicosylic acid | Heneicosanoic acid | CH₃(CH₂)₁₉COOH | C21:0 |
| Behenic acid | Docosanoic acid | CH₃(CH₂)₂₀COOH | C22:0 |
| Tricosylic acid | Tricosanoic acid | CH₃(CH₂)₂₁COOH | C23:0 |
| Lignoceric acid | Tetracosanoic acid | CH₃(CH₂)₂₂COOH | C24:0 |
| Pentacosylic acid | Pentacosanoic acid | CH₃(CH₂)₂₃COOH | C25:0 |
| Cerotic acid | Hexacosanoic acid | CH₃(CH₂)₂₄COOH | C26:0 |
| Carboceric acid | Heptacosanoic acid | CH₃(CH₂)₂₅COOH | C27:0 |
| Montanic acid | Octacosanoic acid | CH₃(CH₂)₂₆COOH | C28:0 |
| Nonacosylic acid | Nonacosanoic acid | CH₃(CH₂)₂₇COOH | C29:0 |
| Melissic acid | Triacontanoic acid | CH₃(CH₂)₂₈COOH | C30:0 |
| Hentriacontylic | Hentriacontanoic | CH₃(CH₂)₂₉COOH | C31:0 |
| acid | acid | | |
| Lacceroic acid | Dotriacontanoic acid | CH₃(CH₂)₃₀COOH | C32:0 |
| Psyllic acid | Tritriacontanoic acid | CH₃(CH₂)₃₁COOH | C33:0 |
| Geddic acid | Tetratriacontanoic | CH₃(CH₂)₃₂COOH | C34:0 |
| | acid | | |
| Ceroplastic acid | Pentatriacontanoic | CH₃(CH₂)₃₃COOH | C35:0 |
| | acid | | |
| Hexatriacontylic | Hexatriacontanoic | CH₃(CH₂)₃₄COOH | C36:0 |
| acid | acid | | |
| Heptatriacontylic | Heptatriacontanoic | CH₃(CH₂)₃₅COOH | C37:0 |
| acid | acid | | |
| Octatriacontylic | Octatriacontanoic | CH₃(CH₂)₃₆COOH | C38:0 |
| acid | acid | | |
| Nonatriacontylic | Nonatriacontanoic | CH₃(CH₂)₃₇COOH | C39:0 |
| acid | acid | | |
| Tetracontylic acid | Tetracontanoic acid | CH₃(CH₂)₃₈COOH | C40:0 |

TABLE 2
Monounsaturated Fatty Acids

| | | | Lipid Numbers |
|---|---|---|---|
| Common | | Molecular | C-Atoms:Double |
| Name | Chemical Name | Formula | Bonds |
| Undecylenic | cis-10-undecenoic acid | C₁₀H₁₉COOH | 11:1 |
| Myristoleic | cis-9-tetradecenoic acid | C₁₃H₂₅COOH | 14:1 |
| Palmitoleic | cis-9-hexadecenoic acid | C₁₅H₂₉COOH | 16:1 |
| Palmitelaidic | trans-9-hexadecenoic acid | C₁₅H₂₉COOH | 16:1 |
| Petroselinic | cis-6-octadecenoic acid | C₁₇H₃₃COOH | 18:1 |
| Oleic | cis-9-octadecenoic acid | C₁₇H₃₃COOH | 18:1 |
| Elaidic | trans-9-octadecenoic acid | C₁₇H₃₃COOH | 18:1 |
| Vaccenic | cis-11-octadecenoic acid | C₁₇H₃₃COOH | 18:1 |
| Gondoleic | cis-9-eicosenoic acid | C₁₉H₃₇COOH | 20:1 |
| Gondolic | cis-11-eicosenoic acid | C₁₉H₃₇COOH | 20:1 |
| Cetoleic | cis-11-docosenoic acid | C₂₁H₄₁COOH | 22:1 |
| Erucic | cis-13-docosenoic acid | C₂₁H₄₁COOH | 22:1 |
| Nervonic | cis-15-tetracosaenoic acid | C₂₃H₄₅COOH | 24:1 |

In certain embodiments, polyunsaturated fatty acids are used to create the microparticles either alone or in mixtures of other fatty acids. Polyunsaturated fats typically have a lower melting point than do their equivalent carbon number saturated fatty acid analogues. Examples of two essential fatty acids are Linoleic acid (C18:2) and α-Linoleic acid (C18:3). The human body cannot make these fatty acids but requires them and must obtain them through dietary intake. The body can metabolize them so they can be used to generate microparticle drug reservoirs but they have multiple double bonds which oxidize easily and may react with some APIs.

TABLE 3
Omega-3 Fatty Acids

| | | Lipid |
|---|---|---|
| Common name | Chemical name | Numbers |
| Hexadecatrienoic acid (HTA) | all-cis 7,10,13- | 16:3 (n-3) |
| | hexadecatrienoic acid | |
| Alpha-linolenic acid (ALA) | all-cis-9,12,15- | 18:3 (n-3) |
| | octadecatrienoic acid | |
| Stearidonic acid (SDA) | all-cis-6,9,12,15- | 18:4 (n-3) |
| | octadecatetraenoic acid | |
| Eicosatrienoic acid (ETE) | all-cis-11,14,17- | 20:3 (n-3) |
| | eicosatrienoic acid | |
| Eicosatetraenoic acid (ETA) | all-cis-8,11,14,17- | 20:4 (n-3) |
| | eicosatetraenoic acid | |
| Eicosapentaenoic acid (EPA, | all-cis-5,8,11,14,17- | 20:5 (n-3) |
| Timnodonic acid) | eicosapentaenoic acid | |
| Heneicosapentaenoic | all-cis-6,9,12,15,18- | 21:5 (n-3) |
| acid (HPA) | heneicosapentaenoic acid | |
| Docosapentaenoic acid (DPA, | all-cis-7,10,13,16,19- | 22:5 (n-3) |
| Clupanodonic acid) | docosapentaenoic acid | |
| Docosahexaenoic acid (DHA, | all-cis-4,7,10,13,16,19- | 22:6 (n-3) |
| Cervonic acid) | docosahexaenoic acid | |
| Tetracosapentaenoic acid | all-cis-9,12,15,18,21- | 24:5 (n-3) |
| | tetracosapentaenoic acid | |
| Tetracosahexaenoic | all-cis-6,9,12,15,18,21- | 24:6 (n-3) |
| acid (Nisinic acid) | tetracosahexaenoic acid | |

TABLE 4
Omega 6 Fatty Acids

| | | Lipid |
|---|---|---|
| Common name | Chemical name | Numbers |
| Linoleic acid (LA) | all-cis-9,12-octadecadienoic acid | 18:2 (n-6) |
| Gamma-linolenic acid | all-cis-6,9,12-octadecatrienoic | 18:3 (n-6) |
| (GLA) | acid | |
| Eicosadienoic acid | all-cis-11,14-eicosadienoic acid | 20:2 (n-6) |
| Dihomo-gamma-linolenic | (DGLA) all-cis-8,11,14- | 20:3 (n-6) |
| acid | eicosatrienoic acid | |
| Arachidonic acid (AA) | all-cis-5,8,11,14-eicosatetraenoic | 20:4 (n-6) |
| | acid | |
| Docosadienoic acid | all-cis-13,16-docosadienoic acid | 22:2 (n-6) |
| Adrenic acid (AdA) | all-cis-7 ,10,13,16- | 22:4 (n-6) |
| | docosatetraenoic acid | |
| Docosapentaenoic acid | all-cis-4,7,10,13,16- | 22:5 (n-6) |
| (Osbond acid) | docosapentaenoic acid | |
| Tetracosatetraenoic acid | all-cis-9,12,15,18- | 24:4 (n-6) |
| | tetracosatetraenoic acid | |
| Tetracosapentaenoic acid | all-cis-6,9,12,15,18- | 24:5 (n-6) |
| | tetracosapentaenoic acid | |

Conjugated fatty acids could also be used alone or in mixtures with other fatty acids to create microparticle drug reservoirs that have desired API solubility/affinity and physical properties.

TABLE 5
Conjugated Fatty Acids

| | | Lipid |
|---|---|---|
| Common name | Chemical name | Number |
| Rumenic acid | 9Z,11E-octadeca-9,11-dienoic acid | 18:2 (n-7) |
| | 10E,12Z-octadeca-10,12-dienoic acid | 18:2 (n-6) |
| α-Calendic acid | 8E,10E,12Z-octadecatrienoic acid | 18:3 (n-6) |
| β-Calendic acid | 8E,10E,12E-octadecatrienoic acid | 18:3 (n-6) |
| Jacaric acid | 8Z,10E,12Z-octadecatrienoic acid | 18:3 (n-6) |
| α-Eleostearic acid | 9Z,11E,13E-octadeca-9,11,13-trienoic | 18:3 (n-5) |
| | acid | |
| β-Eleostearic acid | 9E,11E,13E-octadeca-9,11,13-trienoic | 18:3 (n-5) |
| | acid | |
| Catalpic acid | 9Z,11Z,13E-octadeca-9,11,13-trienoic | 18:3 (n-5) |
| | acid | |
| Punicic acid | 9Z,11E,13Z-octadeca-9,11,13-trienoic | 18:3 (n-5) |
| | acid | |
| Rumelenic acid | 9E,11Z,15E-octadeca-9,11,15-trienoic | 18:3 (n-3) |
| | acid | |
| α-Parinaric acid | 9E,11Z,13Z,15E-octadeca-9,11,13,15- | 18:4 (n-3) |
| | tetraenoic acid | |
| β-Parinaric acid | all trans-octadeca-9,11,13,15-tetraenoic | 18:4 (n-3) |
| | acid | |
| Bosseopentaenoic | 5Z,8Z,10E,12E,14Z-eicosapentaenoic | 20:5 (n-6) |
| acid | acid | |

The drug reservoir microparticles may also be created from animal ester waxes such as bees wax, vegetable waxes, lanolin and derivatives. Animal ester waxes typically contain triacontanyl palitate and mixtures of palmitate, palmitoleate, oleate esters, triglycerides and aliphatic alcohols. Additives such as cholesterol, tryglycerides and aliphatic alcohols may be added to change the physical properties of the microparticles, solubility and affinity of the API to the microparticles and act as a carrier molecule to help the API diffuse out the microparticle.

Mineral waxes, mineral oils and lanolin derivatives may be added to change physical and chemical properties of the drug reservoir particles.

Plant sourced waxes can also be used to create the primary phase of the microparticles. Plant waxes provide an advantage over animal waxes in being easier to control environmental conditions and the same organism (palm or plant) lead to lower batch-to-batch variability. Suitable animal and plant waxes are shown in Table 8. In certain embodiments, the fatty acid microparticle is comprised of a carnauba wax. In further embodiments, the fatty acid microparticle is comprised of a combination of carnauba wax and a fatty acid. In exemplary implementations, the mixture is of carnauba wax and oleic acid, caproic acid, caprylic acid, and/or mixtures of the foregoing.

TABLE 6
Examples of Melting points of fatty acids

| Name | Carbon number | Melting point (° C.) |
|---|---|---|
| Capric Acid | 10 | 32 |
| Lauric Acid | 12 | 43 |
| Myristic Acid | 14 | 54 |
| Palmitic Acid | 16 | 62 |
| Stearic Acid | 18 | 69 |
| Arachidic Acid | 20 | 76 |
| Oleic Acid | 18:1 (n-9) | 16 |
| Linoleic Acid | 18:2 | −5 |

TABLE 7
Example of lowering melting temperature
of a stearic acid oleic acid mixture

| OA:SA | Melting |
|---|---|
| Ratio | Temp ° C. |
| 0.93 | 32 |
| 0.85 | 37 |
| 0.81 | 45 |
| 0.77 | 42 |
| 0.75 | 47 |
| 0.70 | 51 |
| 0.65 | 48 |
| 0.55 | 57 |
| 0.50 | 56 |
| 0.45 | 59 |
| 0.40 | 60 |
| 0.35 | 63 |
| 0.31 | 64 |

TABLE 2
Source of Common Animal and Plant Waxes

| Name | Source |
|---|---|
| Animal Wax | |
| Beeswax | Insects |
| Lanolin | Sheep |
| Chinese wax | Insects |
| Spermaceti | Sperm Whale |
| Shellac | Insect |
| Plant Wax | |
| Bayberry wax | Bayberry fruit |
| Candelilla wax | Shrubs |
| Carnauba wax | Palm Fronds |
| Castor wax | Castor Bean |
| Esparto wax | Esparto Grass |
| Japan wax | Fruit |
| Jojoba Oil | Seed Simmondsia Chinensis |
| Ouricury wax | Palm Fronds |
| Rice bran wax | Rice Bran |
| Soy wax | Soy Oil |
| Tallow tree wax | Tallow Tree Seeds |

Triglycerides are an alternative to pure fatty acids. They have similar physical properties to the pure counterpart and similar solubility of anesthetics. Triglycerides are better tolerated as they are found throughout the body and there are metabolic pathways to absorb and metabolize the lipid. Table 9 lists triglycerides that can be substituted for fatty acids as a lipid drug reservoir particle. In general, even number fatty acid components are selected because the even number fatty acids are more present in tissues. There are some odd number fatty acid triglycerides that are utilized in the body such as triheptanoin found in milk, which are also suitable. Unsaturated fatty acid based triglycerides such as triolein can be used to soften lipid particles or create emulsion droplets if a multiphase formulation is desired. Unsaturated triglycerides are found throughout the body such as tripalmitolein a main component of mammalian fat. Utilizing triglycerides already found in the body increases tolerability and/or reduces likelihood of adverse reactions. In certain embodiments, the concentration of anesthetic agent within a triglyceride microparticle is from about 1-16% by weight.

TABLE 3
Triglycerides

| | Fatty Acid | | Fatty Acid |
|---|---|---|---|
| Common Name | Component | Structure | Saturation |
| Tripropionin | Propanoic acid | C₁₂H₂₀O₆ | C3:0 |
| Tributyrin | Butyric acid | C₁₅H₂₆O₆ | C4:0 |
| Trivalerin | Valeric acid | C₁₈H₃₂O₆ | C5:0 |
| Tricaproin | Caproic acid | C₁₅H₂₆O₆ | C6:0 |
| Triheptanoin | Heptanoic acid | C₂₄H₄₄O₆ | C7:0 |
| Tricaprylin | Caprylic acid | C₂₇H₅₀O₆ | C8:0 |
| Tripelarigonin | Pelargonic acid | C₃₀H₅₆O₆ | C9:0 |
| Tricaprin | Decanoic acid | C₃₃H₆₂O₆ | C 10:0 |
| Triundcylin | Undecanoic acid | C₃₆H₆₈O₆ | C11:0 |
| Trilaurin | Lauric acid | C₃₉H₇₄O₆ | C12:0 |
| Tritridecanoin | Tridecanoic acid | C₄₂H₈₀O₆ | C13:0 |
| Trimyristin | Myristic acid | C₄₅H₈₆O₆ | C14:0 |
| Tripentadecanoin | Pentadecanoic | C₄₈H₉₂O₆ | C15:0 |
| | acid | | |
| Tripalmitin | Palmitic acid | C₅₁H₉₈O₆ | C16:0 |
| Trimargarin | Margaric acid | C₅₄H₁₀₄O₆ | C17:0 |
| Tristearin | Stearic acid | C₅₇H₁₁₀O₆ | C18:0 |
| Triolein | Oleic acid | C₅₇H₁₀₄O₆ | C18:1, |
| | | | cis 9 |
| Trinonadecanoylglycerol | Nonadecanoic | C₆₀H₁₁₆O₆ | C19:0 |
| | acid | | |
| Triarachidin | Arachidic acid | C₆₃H₁₂₂O₆ | C20:0 |
| Triheneicosanoin | Heneicosylic acid | C₆₆H₁₂₈O₆ | C21:0 |
| Trierucin | Erucic Acid | C₆₉H₁₂₈O₆ | C22:cis13, |
| | | | 22:1ω9 |
| Tribehenin | Docosanoic acid | C₆₉H₁₃₄O₆ | C22:0 |
| Tritricosanoin | Tricosanoic acid | C₇₂H₁₄₀O₆ | C23:0 |
| Trilignocerin | Lignoceric acid | C₇₅H₁₄₆O₆ | C24:0 |
| Tripentacosylin | Pentacosylic acid | C₇₈H₁₅₂O₆ | C25:0 |
| Tricerotin | Cerotic acid | C₈₁H₁₅₈O₆ | C26:0 |
| Tricarocerin | Carboceric acid | C₈₄H₁₆₄O₆ | C27:0 |
| Trimontanin | Montanic acid | C₈₇H₁₇₀O₆ | C28:0 |
| Trinonacosylin | Nonacosylic acid | C₉₀H₁₇₆O₆ | C29:0 |
| Trimelissin | Melissic acid | C₉₃H₁₈₂O₆ | C30:0 |
| Trihentriacontylin | Hentriacontylic | C₉₆H₁₈₈O₆ | C31:0 |
| | acid | | |
| Trilacceroin | Lacceroic acid | C₉₉H₁₉₄O₆ | C32:0 |
| Tripsyllin | Psyllic acid | C₁₀₂H₂₀₀O₆ | C33:0 |
| Trigeddin | Geddic acid | C₁₀₅H₂₀₆O₆ | C34:0 |
| Tricerplastin | Ceroplastic acid | C₁₀₈H₂₁₂O₆ | C35:0 |
| Trihexatriacontylin | Hexatriacontylic | C₁₁₁H₂₁₈O₆ | C36:0 |
| | acid | | |
| Triheptatriacontylin | Heptatriacontylic | C₁₁₄H₂₂₄O₆ | C37:0 |
| | acid | | |
| Trioctatriacontylin | Octatriacontylic | C₁₁₇H₂₃₀O₆ | C38:0 |
| | acid | | |
| Trinonatriacontlyin | Nonatriacontylic | C₁₂₀H₂₃₆O₆ | C39:0 |
| | acid | | |
| Tritetracontylin | Tetracontylic acid | C₁₂₂H₂₄₂O₆ | C40:0 |
| Triisopalmitin | Isopalmitic acid | C₅₁H₉₈O₆ | C16:0 |
| Triisostearin | Isostearic acid | C₅₇H₁₁₀O₆ | C18:0 |
| Trilinolein | Linoleic acid | C₅₇H₉₈O₆ | C18:2n-6 |
| Triheptylundecanoin | Heptylundecanoic | C₅₇H₁₁₀O₆ | C18:0 |
| | acid | | |
| Tripalmitolein | Palmitoleic acid | C₅₁H₉₂O₆ | C16:1-8 |
| Triricinolein | Ricinoleic acid | C₅₇H₁₀₄O₉ | C18:1-9, |
| | | | 11-OH |

In certain embodiments, the hydrogel composition contains a plurality of lipid microparticles with varying characteristics in terms of lipid compositions, size, and/or API concentration. In these implementations, mixtures of lipid microparticles are used to improve the elution rate of the drug and tune the elution to produce a steady first order release from the particles. Adjusting the particle volume to carrier phase volume ratio will extend the release duration of the API.

In exemplary implementations, the lipid microparticle is not a liposome.

In certain embodiments, the lipid microparticle is formulated so as to be solid upon being implanted into a subject (e.g. at a temperature of about 37° C.) In further embodiments, the lipid microparticle is formulated so as to be a liquid upon being implanted into a subject, with the effect being that elution rate from such liquid microparticles would increases relative to a solid microparticle with a similar concentration of anesthetic. In still further embodiments, the composition comprises both of the foregoing microparticles so that some microparticles will remain solid and some will become liquid upon implantation into the subject. The relative balance of the two types of microparticles can be adjusted to achieve the desired elution characteristics.

The size of the lipid microparticle ranges in size from about 1 μm to about 20 μm, in certain implementations. In further embodiments, the lipid microparticle ranges in size from about 5 μm to about 15 μm. In certain exemplary embodiments, the lipid microparticle is about 7 μm.

In certain implementations, elution properties of the disclosed composition are affected by the volumetric ratio of the aqueous phase to the lipid phase in the composition. According to certain embodiments, the ratio of aqueous to lipid phase is about 50%-80% aqueous phase volume to about 20%-50% lipid phase volume. According to further embodiments, the ratio of aqueous to lipid phase is about 60%-80% aqueous phase volume to about 20%-40% lipid phase volume. According to still further embodiments, the ratio of aqueous to lipid phase is about 70% aqueous phase volume to about 30% lipid phase volume.

According to certain further embodiments, the composition comprises two are more lipid phases within the aqueous carrier phase. In certain implementations of these embodiments, distributed within the aqueous phase is a lipid microparticle phase, as described previously, and a secondary lipid phase which may take the form of an emulsion within the aqueous phase or a plurality of lipid microparticles from which the API elutes at a faster rate than the primary lipid microparticle phase. The purpose of the aqueous phase is to carry the microparticles and secondary lipid phase and keep these components homogenous throughout the formulation. It provides volume so that an accurate dose can be delivered to the desired tissue site and may contain a salt form of the anesthetic agent (e.g. ropivacaine). The salt form of the anesthetic delivers an upfront burst of drug that matches a similar dose of the saline form of the anesthetic. The primary lipid phase, or drug reservoir microparticle, contains the largest amount of anesthetic in base form and will elute the drug component into the aqueous phase slowly after the upfront burst has eluted from the drug product and into the surrounding tissue. There is a mass transfer limitation due to the solubility of the base form in the aqueous carrier phase and the hydrophilic lipophilic balance (HLB) ratio of the microparticles. The base form has a higher affinity for the lipid phase and the lipid phase will always have some anesthetic present after the elution is complete due to the affinity of the drug for the lipid phase. The secondary lipid phase, or emulsion phase (in some formulations this may be a second type of solid particle), delivers anesthetic at a faster rate than the solid phase microparticles and together they raise the elution rate in the intermediate phase. Once the targeted duration has been met, the elution rate decreases to zero and is below the pharmaceutically effective dose. In certain embodiments, the composition includes and emulsion phase as described above, but without the plurality of solid microparticles.

Suitable lipids for the secondary lipid emulsion phase are any lipid or mixture of lipids that are liquid at 37°. Examples include, but are not limited to stearic acid, oleic acid, caprylic acid, capric acid, lauric acid, palmitic acid, arachidic acid, lignoceric acid, cerotic acid. In certain embodiments, a mixture of stearic acid and oleic acid are the lipids in the lipid emulsion phase. In further embodiments, triglycerides (e.g. trioleate or tripalmitin and trioleate) form the secondary lipid emulsion phase. According to certain embodiments, an emulsifier is used to stabilize the emulsion. Emulsifiers such as TWEEN or other emulsifiers known in the art are suitable.

In certain implementations, anesthetic elution properties of the disclosed composition are affected by the volumetric ratio the two or more lipid phases. According to certain embodiments, the ratio of solid microparticle lipid phase to the emulsion lipid phase is about 50%-75% solid phase volume to about 25%-50% emulsion phase volume. According to certain embodiments, the ratio of solid microparticle lipid phase to the emulsion lipid phase is about 66% solid phase volume to about 34% emulsion phase volume.

### Methods of Formulating Lipid Microparticle Hydrogel Composition

According to certain embodiments, lipid microparticles are generated by agitating a solution of fatty acid phase containing API in a much larger volume aqueous phase. The preferred ratio of aqueous to lipid phase is 95%-99.5% aqueous phase to 0.5%-5% lipid phase. It is preferred that the aqueous phase be saturated with the API that is present in the lipid phase. In certain embodiments, a salt in >25 mmol concentration is present in the aqueous phase preferably between 25 and 150 mmol, more preferred to be between 45 and 65 mmol. Tyramine substituted hyaluronic acid is present in the aqueous phase at 0.1% to 4% preferably between 0.1 to 1% and specifically at 0.5% concentration. The two-phase mixture is agitated and cooled until microparticles are generated. The particles are concentrated using a centrifuge, filter or settling tank and the aqueous phase decanted leaving the microparticles behind. Additional aqueous phase containing tyramine substituted hyaluronic acid and horse radish peroxidase is added to the free microparticles and the particles are suspended in the solution at a volume ratio of 30% lipid phase to 70% aqueous phase. A hydrogel is formed with the addition of hydrogen peroxide. The hydrogel maintains particle separation and allows for easy delivery via syringe.

According to certain embodiments, formulations with two or more lipid phases (e.g. lipid microparticle and emulsion) the formulation can be prepared as in the preceding paragraph except that prior to the addition of microparticles to the aquas phase, anesthetic dissolved in a liquid lipid phase (in certain embodiments a mixture of stearic acid and oleic acid) and mixed vigorously with the aqueous phase until an emulsion is formed. Following the formation of the emulation, the lipid microparticles are added as described previously.

Without wishing to bound to theory, it is believed that the zeta potential is increased by adding the salt (e.g., NaCl) to the aqueous phase, causing the surface charge to increase and cause the particles to repel each other allowing smaller diameter particles to form and preventing coalescing particles from forming larger particles prior to solidification. In certain implementations, the hydrogel comprises between 10 mM and about 70 mM salt. In further implementations, salt concentration is between about 25 mM and about 50 mM salt. In further implementations, the hydrogel comprises at least about 50 mM salt. In certain aspects, the salt is NaCl. As will be appreciated by those skilled in the art, other salts are possible.

In certain implementations of the disclosed composition, the anesthetic agent comprises ropivacaine. In exemplary aspects, the ropivacaine is present in the lipid microparticles in an amount of from about 1 to about 25%. In further embodiments, where the lipid microparticles are comprised of triglycerides,

According to certain alternative embodiments, anesthetic unbound by the plurality of lipid microparticles is dispersed throughout the hydrogel. According to these embodiments, the API dispersed throughout the hydrogel provides for an immediate burst dose, while the API bound in the lipid microparticles provides for extended sustained release.

In certain implementations, the composition further comprises a radiopaque contrast agent.

Further disclosed herein is a method of treating post-operative pain in a subject in need thereof comprising administering to the subject and effective amount of a composition comprising an immiscible carrier phase and a plurality of lipid microparticles dispersed within the immiscible carrier phase comprising an anesthetic agent. In certain implementations, the immiscible carrier phase is a hydrogel, a viscous liquid, a stable emulsion, or a cream.

In exemplary implementations, the immiscible carrier phase is a hydrogel (e.g., a hydrogel comprised of tyramine substituted hyaluronic acid).

In certain embodiments, the anesthetic is selected from: ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, or a pharmaceutically acceptable salt thereof, or a mixture thereof. In certain implementations, the anesthetic is ropivacaine. In certain alternative embodiments the anesthetic in bupivacaine.

In certain implementations of the disclosed method, the composition is administered to the subject and is delivered near a never or nerve bundle of a subject. In exemplary embodiments, the nerve or nerve bundle innervates the surgical incision area of the subject. The composition may be delivered by way of a syringe or hypodermic needle, other delivery methods known in the art. In exemplary implementations of the disclosed method, the administration of the composition as described herein provides pain relief for about 72 hours or more.

Also provided herein are kits of pharmaceutical formulations containing the disclosed compounds or compositions. The kits may be organized to indicate a single formulation or combination of formulations. The composition may be sub-divided to contain appropriate quantities of the compound. The unit dosage can be packaged compositions such as packeted powders, vials, ampoules, prefilled syringes or sachets containing liquids.

The compound or composition described herein may be a single dose or for continuous or periodic discontinuous administration. For continuous administration, a kit may include the compound in each dosage unit. For periodic discontinuation, the kit may include placebos during periods when the compound is not delivered. When varying concentrations of the composition, the components of the composition, or relative ratios of the compound or other agents within a composition over time is desired, a kit may contain a sequence of dosage units.

The kit may contain packaging or a container with the compound formulated for the desired delivery route. The kit may also contain dosing instructions, an insert regarding the compound, instructions for monitoring circulating levels of the compound, or combinations thereof. Materials for performing using the compound may further be included and include, without limitation, reagents, well plates, containers, markers or labels, and the like. Such kits are packaged in a manner suitable for treatment of a desired indication. Other suitable components to include in such kits will be readily apparent to one of skill in the art, taking into consideration the desired indication and the delivery route. The kits also may include, or be packaged with, instruments for assisting with the injection/administration or placement of the compound within the body of the subject. Such instruments include, without limitation, syringe, pipette, forceps, measuring spoon, eye dropper or any such medically approved delivery means. Other instrumentation may include a device that permits reading or monitoring reactions in vitro.

The compound or composition of these kits also may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In one embodiment, the package is a labeled blister package, dial dispenser package, or bottle.

Further disclosed herein is a composition for sustained release of an active pharmaceutical ingredient (API) comprising: a hydrogel; and a plurality of lipid microparticles dispersed within the hydrogel comprising the API. In certain embodiments the API is a chemotherapeutic composition. In further embodiments, the API is a motion sickness drug. In exemplary implementations, the motion sickness drug is meclizine or dimenhydrinate. In further implementations, the API is selected from NSAIDS, steroids, biologics such as antibodies, hormones.

Further disclosed herein are at least the following compositions.

Composition 1 is a composition for treating post-surgical pain comprising: an aqueous carrier; and a lipid phase comprising an anesthetic agent, the lipid phase dispersed within the aqueous carrier.

2. The composition of composition 1, wherein the aqueous carrier is hydrogel comprised of tyramine substituted hyaluronic acid, wherein the hydrogel is formed through di-tyramine crosslinking and wherein the degree of tyramine substitution of hyaluronic acid hydroxyl groups is about 0.5% to about 3%.

3. The composition of composition 1, wherein the lipid phase comprises a plurality of lipid microparticles.

4. The composition of composition 1, wherein the lipid phase is emulsified within the aqueous carrier.

5. The composition of composition 3, wherein a salt form of the anesthetic unbound by the plurality of lipid microparticles is dissolved in the aqueous carrier.

6. The composition of claim 5, wherein the volumetric ratio between the aqueous carrier and the lipid microparticles is from about 70-80 the aqueous carrier to about 30-20 lipid microparticles.

7. The composition of any of compositions 3-6, wherein the lipid microparticles comprise one or more fatty acids having an even number of carbons.

8. The composition of any of compositions 3-7, wherein the lipid microparticles comprise one or more fatty acids having an odd number of carbons.

9. The composition of any of compositions 3-8, wherein the one or more fatty acids are chosen from: stearic acid, oleic acid, myristic acid, caprylic acid, capric acid, lauric acid, palmitic acid, arachidic acid, lignoceric acid, cerotic acid, and mixtures of the forgoing and wherein the melting point of the lipid microparticle is above 37° C.

10. The composition of any of compositions 3-9, wherein the one or more fatty acids comprise a mixture of steric acid and oleic acid and wherein the ratio of steric acid to oleic acid is about 90:10.

11. The composition of composition 7, wherein in the lipid microparticles comprise about 12% myristic acid, about 32% palmitic acid, about 10% stearic acid, and about 10% oleic acid.

12. The composition of composition, wherein the lipid microparticles comprise a mixture of lauric acid and caprylic acid, caproic acid, and/or oleic acid.

13. The composition of composition 3, wherein the lipid microparticle comprises a paraffin, a triglyceride, and/or a wax.

14. The composition of composition 13, wherein the lipid microparticles comprise a mixture of carnauba wax and caprylic acid, caproic acid, and/or oleic acid.

15. The composition of compositions 3-14, wherein the plurality of lipid microparticles comprises a first plurality of lipid microparticles and a second plurality of lipid microparticles and wherein the first plurality of lipid microparticles is solid at about 37° C. and the second plurality of lipid microparticles is liquid at 37° C.

16. The composition of any previous composition, wherein the lipid microparticle is not a liposome.

17. The composition of any previous composition, wherein the lipid microparticle ranges in size from about 1 μm to about 20 μm.

18. The composition of any previous composition, wherein the lipid microparticle ranges in size from about 4 μm to about 8 μm.

19. The composition of any previous composition, wherein the anesthetic agent comprises ropivacaine.

20. The composition any previous composition, wherein the ropivacaine is present in the lipid microparticles in an amount of from about 1 to about 25% by weight.

21. A composition for treating post-surgical pain comprising: an aqueous carrier; a first lipid phase comprising a plurality of lipid microparticles comprising an anesthetic agent and dispersed within the aqueous carrier; and a second lipid phase comprising an anesthetic agent dissolved in one or more lipids and emulsified into to the aqueous phase.

22. The composition of composition 21, wherein a salt form of the anesthetic agent, not present in the first lipid phase or the second lipid phase, is dissolved in the aqueous carrier.

23. The composition of compositions 21-22, wherein the one or more lipids of the second lipid phase is one or more fatty acids and wherein the second lipid phase is emulsified into the aqueous phase.

24. The composition of compositions 21-23, wherein the one or more fatty acids of the second lipid phase are a mixture of stearic acid and oleic acid.

25. The composition of compositions 21-24, wherein the volumetric ratio of the first lipid phase and the second lipid phase is about 66:34.

26. The composition of any of compositions 21-25, wherein the lipid microparticles comprise one or more fatty acids having an even number of carbons.

27. The composition of any of compositions 21-25, wherein the lipid microparticles comprise one or more fatty acids having an odd number of carbons.

28. The composition of any of compositions 21-27, wherein the one or more fatty acids are chosen from: stearic acid, oleic acid, myristic acid, caprylic acid, capric acid, lauric acid, palmitic acid, arachidic acid, lignoceric acid, cerotic acid, and mixtures of the forgoing and wherein the melting point of the lipid microparticle is above 37° C.

29. The composition of any of compositions 21-28, wherein the one or more fatty acids comprise a mixture of steric acid and oleic acid and wherein the ratio of steric acid to oleic acid is about 90:10.

30. The composition of compositions 21-29, wherein in the lipid microparticles comprise about 12% myristic acid, about 32% palmitic acid, about 10% stearic acid, and about 10% oleic acid.

31. The composition of compositions 21-30, wherein the lipid microparticles comprise a mixture of lauric acid and caprylic acid, caproic acid, and/or oleic acid.

32. The composition of compositions 21-31, wherein the lipid microparticle comprises a paraffin, a triglyceride, and/or a wax.

33. The composition of composition 32, wherein the lipid microparticles comprise a mixture of carnauba wax and caprylic acid, caproic acid, and/or oleic acid.

34. The composition of compositions 21-33, wherein the plurality of lipid microparticles comprises a first plurality of lipid microparticles and a second plurality of lipid microparticles and wherein the first plurality of lipid microparticles is solid at about 37° C. and the second plurality of lipid microparticles is liquid at 37° C.

35. A composition for sustained release of an active pharmaceutical ingredient (API) comprising: a hydrogel; and a plurality of lipid microparticles dispersed within the hydrogel comprising the API.

36. The composition of composition 35, wherein the API is a chemotherapeutic composition.

37. The composition of composition 35, wherein the API is a motion sickness drug.

38. The composition of composition 35, wherein the motion sickness drug is meclizine or dimenhydrinate.

39. The composition of composition 35, wherein the API is selected from NSAIDS, steroids, biologics such as antibodies, hormones.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1: Effect of Aqueous Phase Salt Concentration of Lipid Microparticle Size

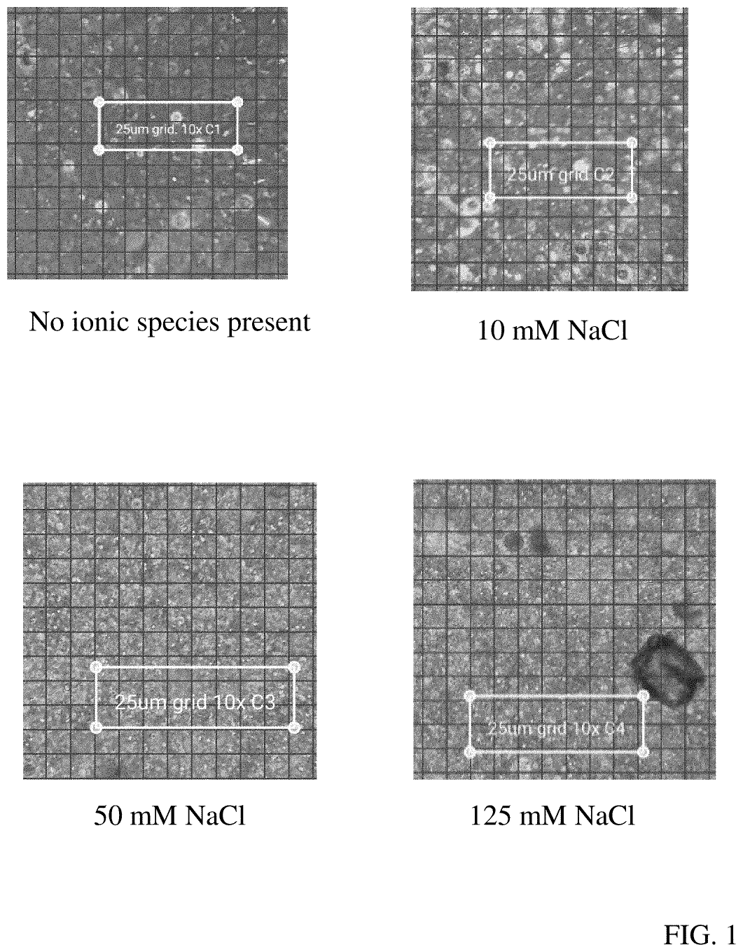

Lipid microparticles were prepared by mixing liquid fatty acid phase containing the API dissolved in the phase with a saline aqueous phase of the same temperature and then rapidly cooling to fix the particle diameter and prevent the fatty acid droplets from coalescing into larger particles. To assess the effect of aqueous phase ionic content on particle size, samples were prepared at varying ionic concentrations. As shown in FIG. 1, with no ionic species present in the solution, large amounts of fatty acid coalesced into large globules and few microparticles generated. 10 mM NaCl generated more microparticles and some smaller macro particles, with some large particles coalesced in vial. 50 mM NaCl increases number of microparticles and volume of microparticles generated from initial fatty acid volume. This indicates that adding NaCl keeps the particles from coalescing and forming large particles. Adding an ionic species to the solution also reduces particle diameter to <10 um. Increasing concentration of NaCl to 125 mM did not increase number of microparticles and macroparticle numbers increased.

### Example 2: Preparation of Ropivacaine Lipid Microparticle Hydrogel Composition

Samples of Ropivacaine lipid microparticle hydrogel composition were prepared according to the following procedure. All actions requiring open containers or transfers between containers occurred in the cleaned and sterilized (10% bleach cleaning solution) isolation hood. Ropivacaine base was dissolved in Stearic Acid at a concentration of 93.5 mg/mL lipid phase at 75° C. in a sterile sealed tube. Once the lipid had completely dissolved the ropivacaine, the liquid lipid phase was sterile filtered through a 75° C. preheated 0.2 um syringe filter and transferred to a preheated sterile centrifuge tube where it was kept at 75° C. until ready to mix with the 75° C. preheated aqueous hydrogel phase. The THA powder (0.25%) and 50 mM sodium chloride were dissolved in sterile filtered RO water and allowed to sit for up to 6 hours until the THA powder was completely dissolved. This final solution was double sterile filtered into a new sterile centrifuge tube and then heated to 75° C. The lipid phase and aqueous phase were then combined, sealed, and shaken vigorously to create a uniform suspension of approximately Sum lipid particles suspended in the THA solution. The resulting suspension was filtered through a sterile 0.2 micron cellulose filter media to collect the microparticles. The resulting filtrate was not used and retained for future analysis. The microparticles were dried in the isolation hood for 12 hours and then transferred into a sterile tube and sealed until the final formulation was ready. A carrier phase HRP buffer was added to a tube and mixed with 0.25% THA and 14.28 mg/mL ropivacaine HCl. The solution was allowed to sit for 12 hours refrigerated until the THA component was completely dissolved. The final solution was sterile filtered into a final formulation tube in the isolation hood. 1.5495 g of microparticles was added to 3.5102 g HRP/THA buffer containing 14.28 mg/mL ropivacaine HCL and crosslinked with 0.1640 g 3% H₂O₂. The gel mass was transferred to a sterile 10 mL syringe and filled into 1 mL syringes.

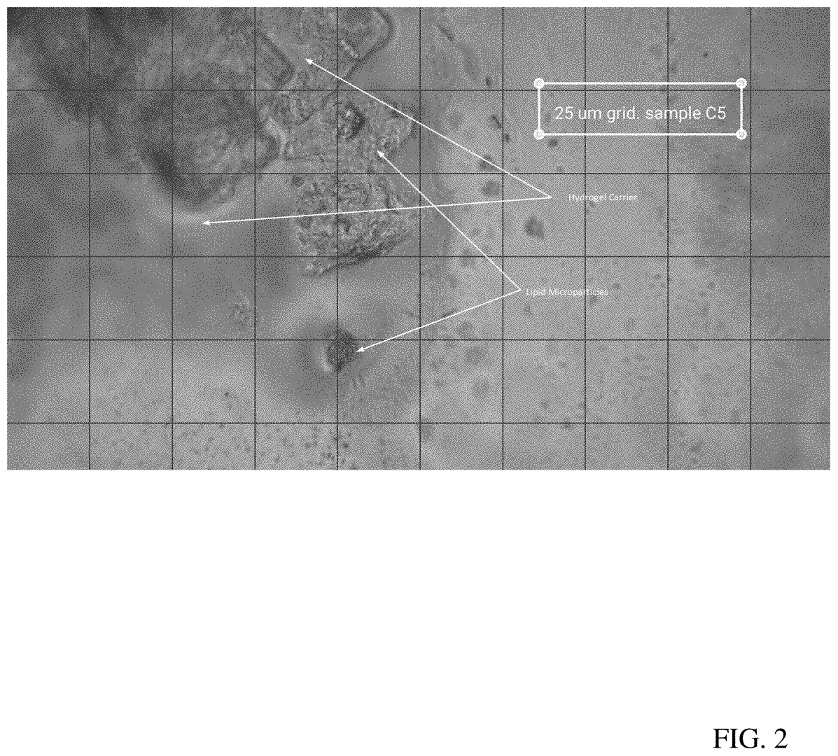

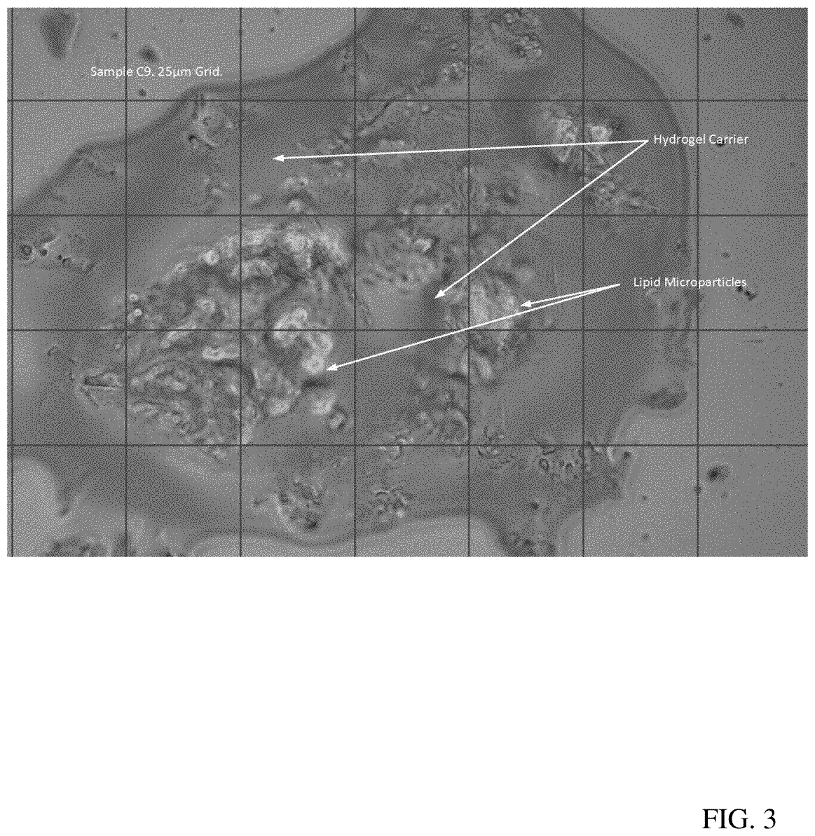

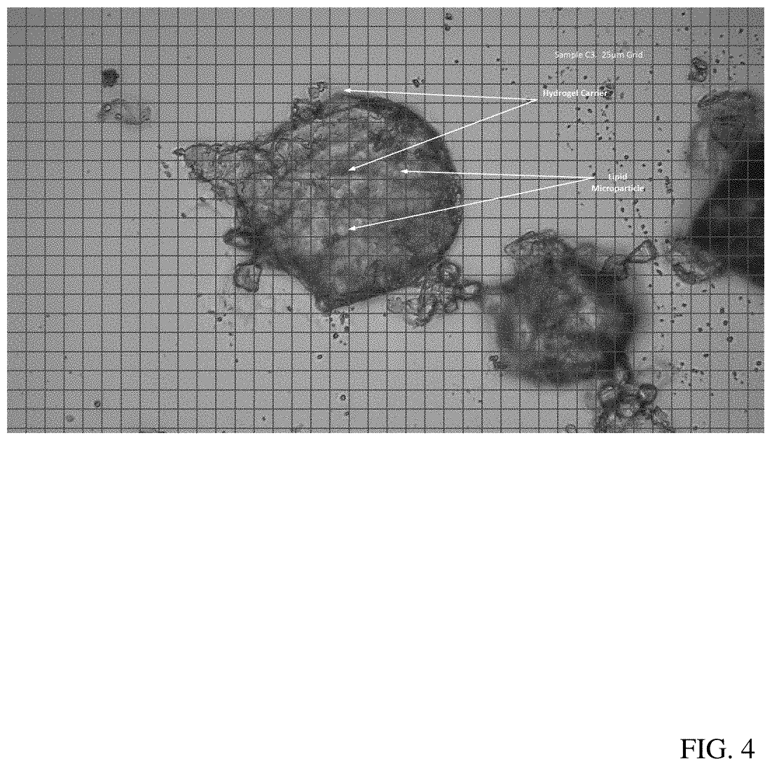

FIGS. 2-4 show representative images of the ropivacaine lipid microparticle hydrogel composition, with key features identified by arrows.

In order to test the effects of the disclosed compositions in vivo, animal studies were performed. Briefly, Sprague-Dawley rats (n=6 per treatment group) received an injection of the composition of interest proximal to the sciatic nerve. Blood samples were collected via indwelling jugular catheter and analyzed to determine blood plasma concentrations of the relevant anesthetic. The results of these studies are shown in examples below.

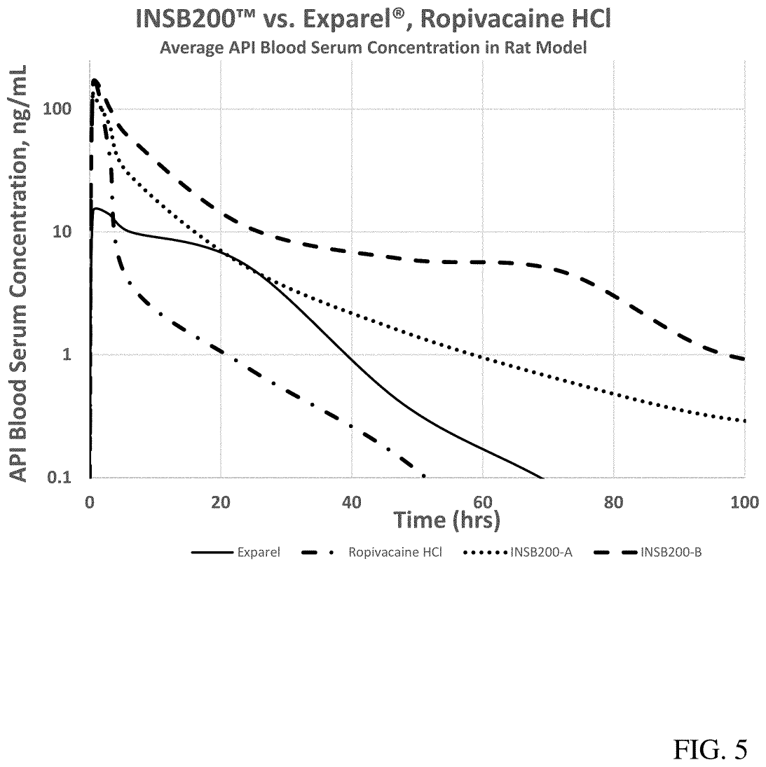

FIG. 5 shows the results of studies in rats comparing serum API concentrations in animals administered the two formulations of the ropivacaine lipid microparticle hydrogel composition (INSB200-A and INSB200B), Ropivacaine HCL, and a commercially available bupivacaine sustained release formulation (Exparel). The formulation INSB200-A is 30% lipid microparticle by volume (6 mL lipid, 14 mL aqueous). The lipid microparticle are stearic acid containing 70 mg/mL Ropivacaine base, 14.2 mg/mL Ropivacaine HCl in the aqueous carrier.

The formulation INSB200-B, is 30% lipid microparticle particles by volume. There are two lipid microparticle populations present in a 34:66 ratio of 90/10 Stearic/oleic acid containing 110 mg/mL Ropivacaine base and a stearic acid lipid microparticle containing 110 mg/mL Ropivacaine base. The aqueous carrier phase contains 14.2 mg/mL Ropivacaine HCl.

Animals administered both INSB200-A and INSB200-B showed elevated and sustained plasma API levels that were superior to that achieved with a comparable dose of Exparel.

### Example 3

One system evaluated is an aqueous carrier phase with a lipid-based drug reservoir. In the case for INSB200, the API is a nonpolar amide anesthetic ropivacaine. An effective product delivers an upfront burst of API to match a standard nerve block and produces a robust nerve block within a short time without additional injections of anesthetic. The anesthetic elutes at a slower rate after a 24-36 hour period post-surgical procedure so that motor function returns and yet continues to elute at an effective dose to reduce or eliminate post-surgical pain. A two-phase system delivers an upfront burst with ropivacaine HCl in the aqueous carrier phase and then reduces the elution rate until it reaches a steady state that maintains a local concentration of API that effectively treats or minimizes pain.

Three formulations were prepared with an equivalent dose to a 200 mg Naropin block of ropivacaine in the aqueous phase. Ropivacaine base was dissolved in liquid stearic acid at a target concentration of 110 mg/g lipid phase at 75° C. in a sterile sealed tube. Batch 1 contained 0.1524 g ropivacaine base in 1.4423 g stearic acid and batch 2 contained 0.1540 g ropivacaine base in 1.4941 g stearic acid. Both batches were then the mixture heated to 75° C. The liquid lipid batches were agitated frequently until the ropivacaine had completely dissolved in the stearic acid. Once the ropivacaine was completely dissolved the liquid lipid phase was sterile filtered through a 75° C. preheated 0.2 μm syringe filter and transferred to a preheated sterile centrifuge tube where it was kept at 75° C. until ready to mix with the 75° C. preheated aqueous hydrogel phase. The microparticle buffer solution was prepared ahead of time by adding the THA powder (0.25% by weight total buffer) to a sterilized 50 mM sodium chloride solution in reverse osmosis water and allowed to mix for up to 6 hours until the THA powder was completely dissolved. The microparticle saline contained 0.5595 g ropivacaine base, 0.25 g THA, 2.922 g NaCl and 1 L sterile water. Once the THA was completely dissolved, the microparticle saline was sterile filtered into a new sterile tube. Two batches of microparticles were created, batch 1 added 1.5947 g stearic acid/Ropivacaine base to 48.4747 g THA MP saline and batch 2 added 1.6494 g of the stearic acid/Ropivacaine base lipid to 48.5275 g saline. The lipid phase and aqueous phase were then quickly combined, sealed, and agitated vigorously while allowing to cool to room temperature to create a uniform suspension of approximately 0-100 μm lipid particles suspended in the THA solution. The resulting suspension was filtered through a sterile 0.2-micron cellulose filter media to collect the microparticles and the saline filtrate discarded. The microparticles were dried in the sterile isolation hood for 12 hours and then transferred into a sterile tube and sealed until the final formulation was ready. 45.0010 g of HRP buffer (53 mg horse radish peroxidase in 1 L water) was added to a tube and mixed with 0.1130 g THA and 0.6447 g ropivacaine HCl. The solution was allowed to sit for 12 hours refrigerated until the THA component was completely dissolved. The carrier phase final solution was then sterile filtered into a final formulation tube in the isolation hood. 1.5643 g of microparticles was added to 3.5902 g HRP/THA buffer containing 14.31 mg/mL ropivacaine HCL and crosslinked with 0.1753 g 3% H₂O₂. The resulting gel mass was transferred to a sterile 10 mL syringe and filled into 1 mL syringes.

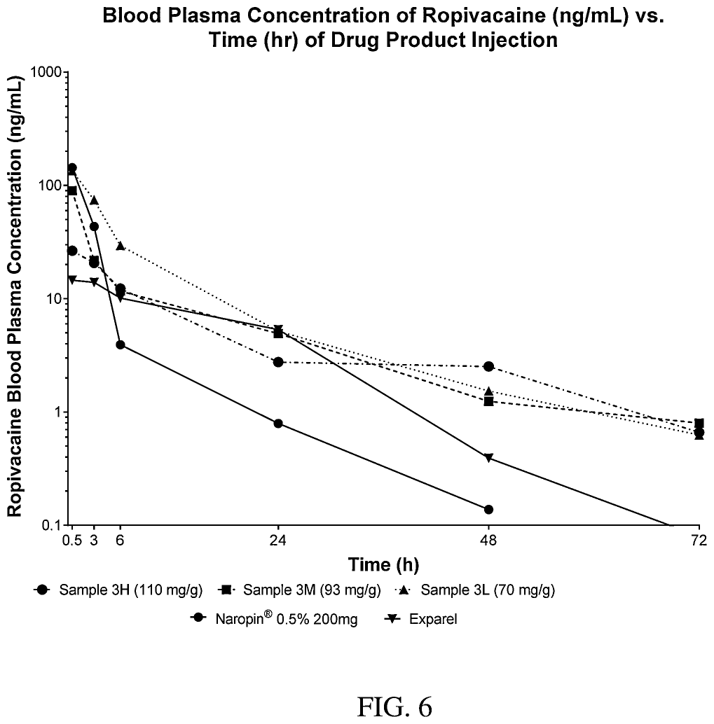

As shown in FIG. 6, all formulations match the Naropin 200 mg injection Cₘₐₓ concentration quickly in the first half hour and then level off at an anesthetic rate for at least 72 hrs. Two controls were used to measure the formulation effectiveness. The first positive control was 0.5% Naropin to show where the formulation needs to deliver a similar dose of ropivacaine in the first 0.5-1 hour. All three formulations delivered an upfront dose that matches the Naropin control. The second positive control was Exparel, a liposomal bupivacaine sustained release anesthetic. The three formulations continue to deliver a higher amount of ropivacaine after 24 hrs than Exparel. Bupivacaine and ropivacaine have similar chemical structures and properties and differ by one methyl group.

All three formulations deliver a similar amount of ropivacaine at 72 hours suggesting that they all contain enough ropivacaine to continue to saturate the carrier phase for over 72 hours. The lipid phase will hold on to some drug and these formulations reach this equilibrium concentration around the same time. This shows that higher concentrations in the range of 70-110 mg ropivacaine/g reservoir in the lipid reservoir particles do not contribute to either the Cₘₐₓ or the overall elution rate.

A sustained release peripheral nerve block product should produce a fast onset robust nerve block that reduces its elution rate to a point where motor function returns to the target limb yet high enough to maintain good analgesia to the target limb. The analgesia should last for at least 72 hours.

As shown in FIG. 6, a two-phase system with a 200 mg equivalent dose in the aqueous carrier phase and fatty acid based lipid microparticles provides an upfront burst and sustained release at a higher dose rate than existing products.

### Example 4

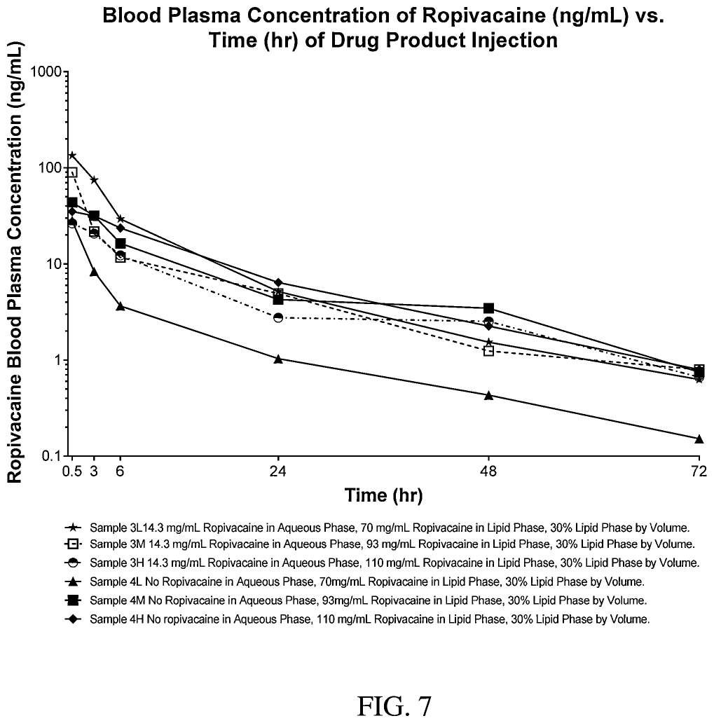

FIG. 7 compares the effect of removing the aqueous phase ropivacaine salt and shows that the elution rate is similar for the formulations that contain >93 mg/g reservoir ropivacaine base. The upfront burst phase releases the ropivacaine HCl quickly so that after 6 hours the formulations match the elution rates of the ropivacaine HCl free formulations. The formulation with below 93 mg/g reservoir that does not contain ropivacaine HCl in the carrier phase is an outlier and quickly reduces elution rate. There is likely an equilibrium level just below 70 mg/g in the lipid microparticles at which the affinity of ropivacaine to the lipid phase is too high for the ropivacaine to be released from the reservoir. This is the lower limit of elution below which the microparticles will not release the API. Formulations must contain more ropivacaine than this lower limit to provide effective sustained release of the API. The 70 mg/g reservoir formulation with the ropivacaine HCl in the carrier phase was likely able to suppress elution as the carrier phase was pre-saturated with ropivacaine so it was able to have a similar elution rate to the higher concentration formulations. This suggests that 70 mg ropivacaine/g reservoir is close to the lower limit. The higher concentration lipid phase with aqueous ropivacaine shows the upfront burst better than a lower concentration.

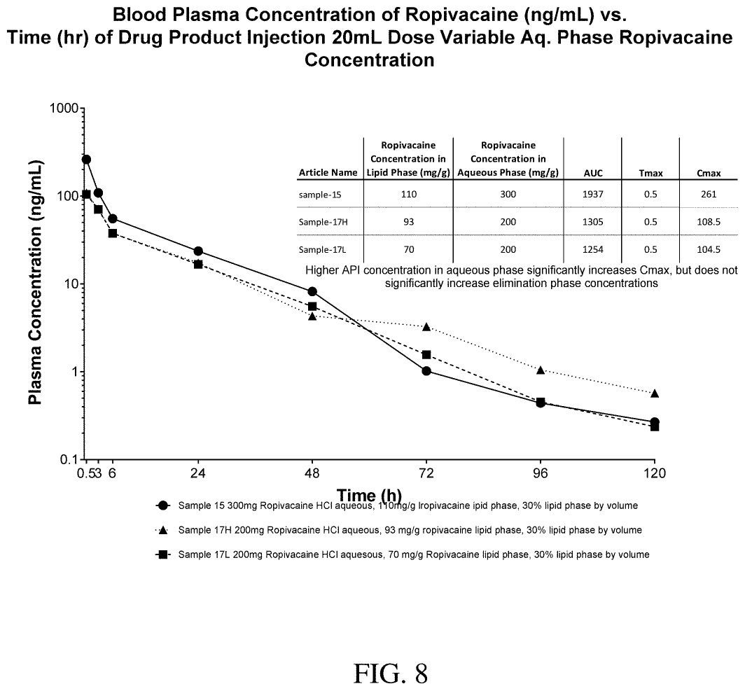

FIG. 8 shows how increasing the aqueous phase ropivacaine HCl concentration will increase Cₘₐₓ but it does not change the overall elution curve significantly. This suggests that the ropivacaine HCl within the aqueous phase elutes independently of the lipid phase components.

A preferred ratio of lipid phase to aqueous phase is <30% lipid phase and greater than 20% lipid phase by volume. Increasing the lipid phase concentration actually reduces the elution rate in some formulations.

### Example 5

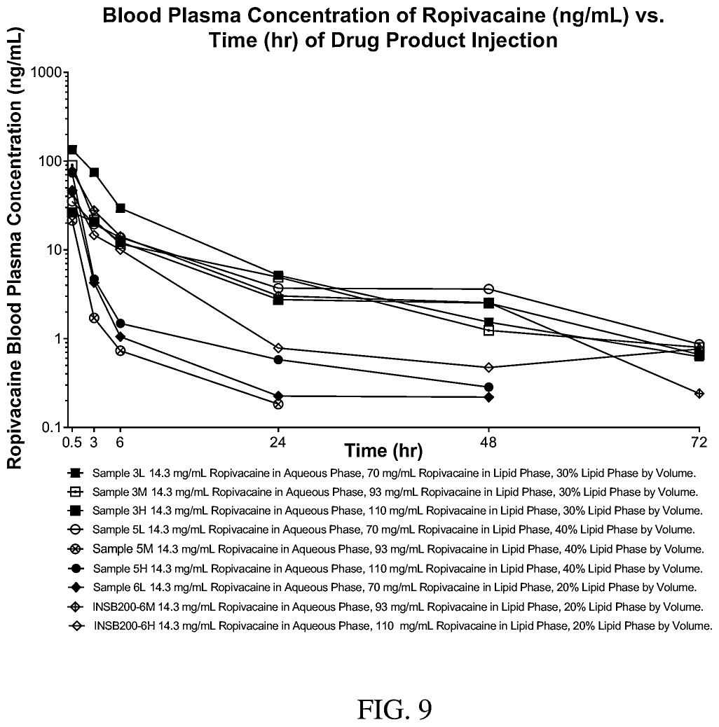

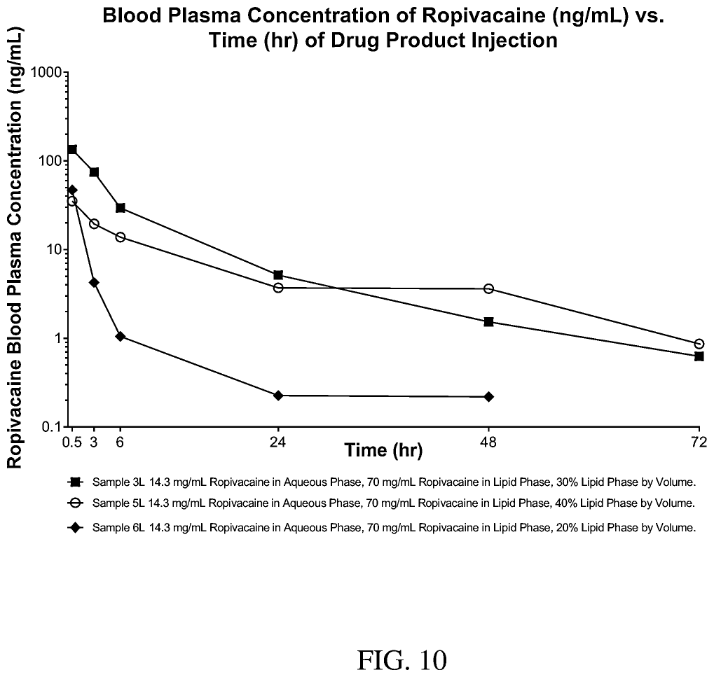

FIGS. 9 and 10 show comparisons for formulations with different volumes of lipid phase. The low loaded drug reservoir formulations performed similarly except that the 20 percent formulation, sample 6 L eluted ropivacaine at a significantly lower rate than the higher loaded formulations. This may be due to the fact that all three formulations are close to the minimum concentration that allows elution and that the 20% volume was out of available ropivacaine before the other two formulations and dropped off quickly.

### Example 6

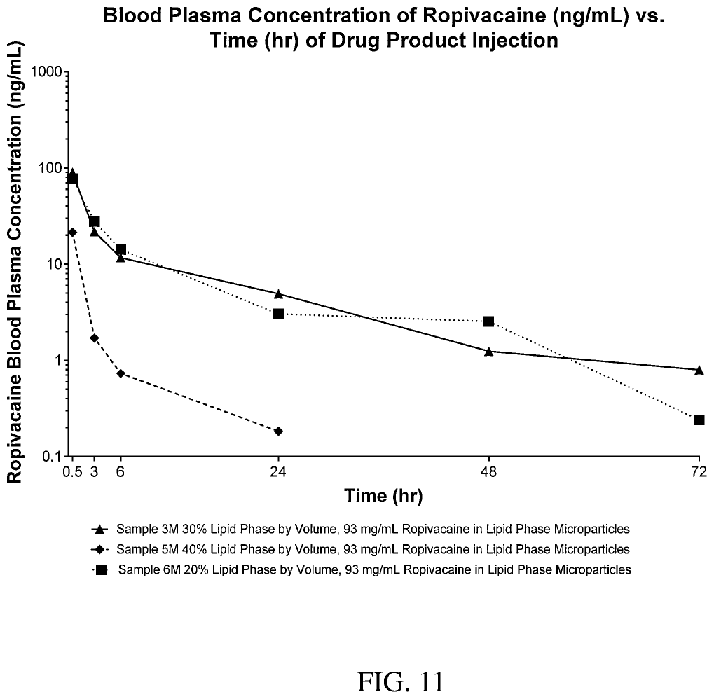

FIG. 11 shows the comparison of midlevel loading of ropivacaine in the lipid drug reservoir. In this set the 40% lipid phase volume formulation performed worse than the other formulations.

### Example 7

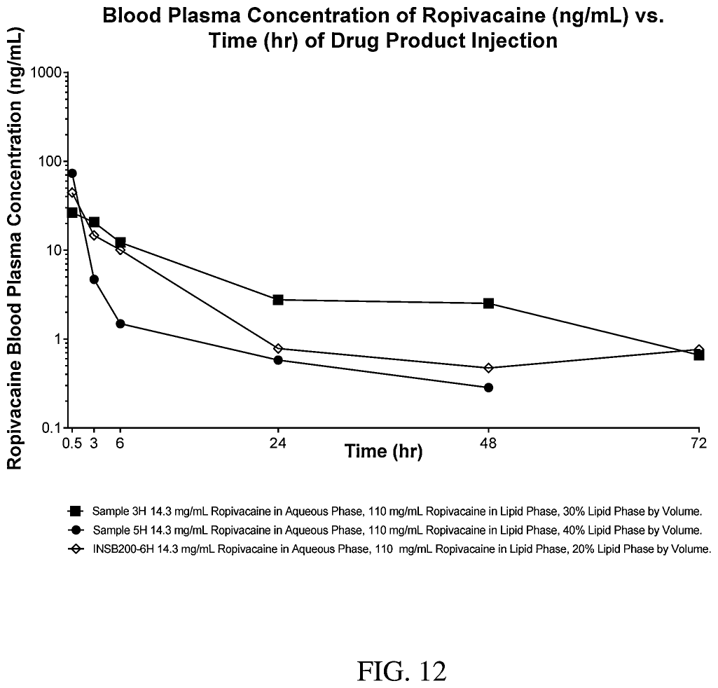

FIG. 12 compares the high loaded ropivacaine concentration in the lipid phase reservoir to each other based on the lipid phase percent volume. Again, the formulation with 30% lipid volume performs better than the other volume percent formulations. This suggests that 30% lipid volume is preferred for sustained release formulations. These formulations may be suitable for other applications where a lower drug dose is necessary or faster release, but for the amide anesthetic the 30% lipid volume drug reservoir formulation is preferred.

### Example 8

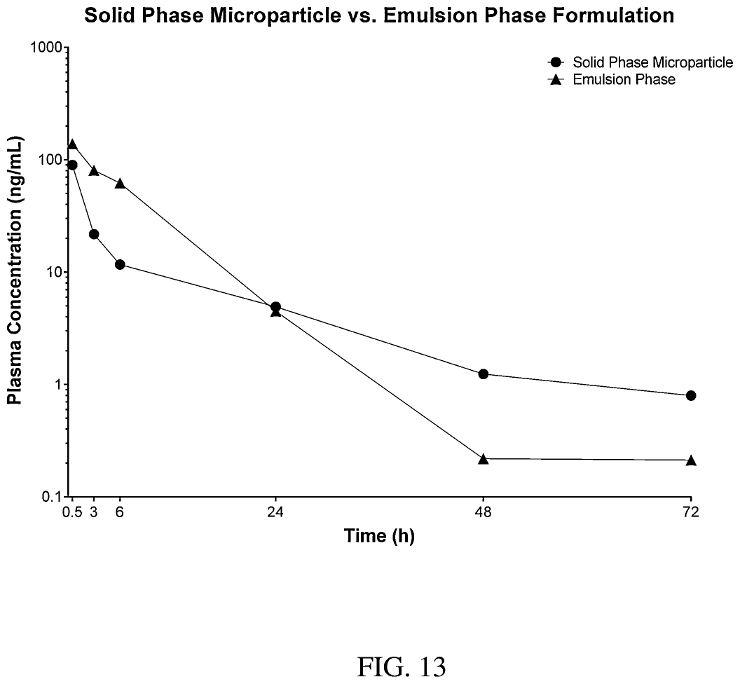

FIG. 13 compares the elution rates of an emulsion phase drug reservoir formulation to a solid phase drug reservoir formulation. Ropivacaine base was dissolved in liquid stearic acid at a target concentration of 110 mg/g lipid phase at 75° C. in a sterile sealed tube. Microparticle lipid phase contained 0.2032 g ropivacaine base in 1.8664 g stearic acid and the emulsion phase lipid phase contained 0.1014 g ropivacaine base in 0.0960 g stearic acid and 0.8393 g oleic acid. Both batches were then heated to 75° C. The liquid lipid batches were agitated frequently until the ropivacaine had completely dissolved in the lipid phase. Once the ropivacaine was completely dissolved the liquid lipid phase was sterile filtered through a 75° C. preheated 0.2 μm syringe filter and transferred to preheated sterile centrifuge tubes where it was kept at 75° C. until ready to mix with the 75° C. preheated aqueous hydrogel phase. The microparticle buffer solution was prepared ahead of time by adding the THA powder (0.25% by weight total buffer) to a sterilized 50 mM sodium chloride solution in reverse osmosis water and allowed to mix for up to 6 hours until the THA powder was completely dissolved. The microparticle saline contained 0.5595 g ropivacaine base, 0.25 g THA, 2.922 g NaCl and 1 L sterile water (same batch as 3 and 4). Once the THA was completely dissolved, the microparticle saline was sterile filtered into a new sterile tube. One batch of microparticles was created, that added 2.0696 g stearic acid/Ropivacaine base to 48.5626 g THA MP saline. The microparticle lipid phase and aqueous phase were then quickly combined, sealed, and agitated vigorously while allowing to cool to room temperature to create a uniform suspension of approximately 0-100 um lipid particles suspended in the THA solution. The resulting suspension was filtered through a sterile 0.2 micron cellulose filter media to collect the microparticles and the saline filtrate discarded. The microparticles were dried in the sterile isolation hood for 12 hours and then transferred into a sterile tube and sealed until the final formulation was ready. 45.0010 g of HRP buffer (53 mg horse radish peroxidase in 1 L water) was added to a tube and mixed with 0.1130 g THA and 0.6447 g ropivacaine HCl. The solution was allowed to sit for 12 hours refrigerated until the THA component was completely dissolved. The carrier phase final solution was then sterile filtered into a final formulation tube in the isolation hood. 0.5148 g of secondary lipid phase (emulsion phase) was added to 3.6100 g of HRP/THA buffer and agitated vigorously to form and emulsion. 1.0424 g of microparticles was added to the now emulsion buffer containing and crosslinked with 0.1757 g 3% H₂O₂. The resulting gel mass was transferred to a sterile 10 mL syringe and filled into 1 mL syringes. Without wishing to be bound to any specific theory, it is thought that an emulsion phase should release the ropivacaine faster as diffusion through a liquid is faster than in a solid. In fact, this example shows a faster elution rate for the first 24 hours where the curves diverge and the solid phase elution rate becomes higher. Combining the two types of reservoirs improves the overall performance of the drug product.

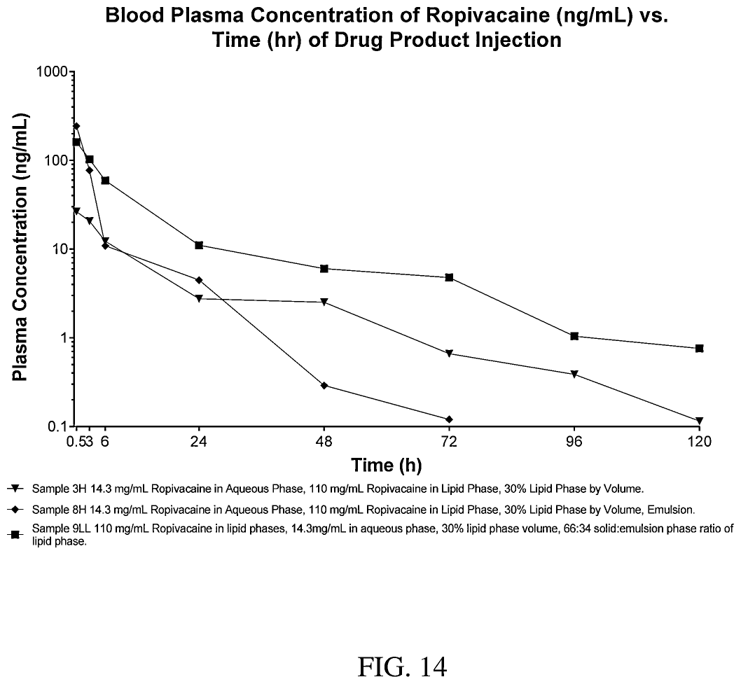

FIG. 14 shows how the formulation improves by combining the two formulations in 66% solid phase reservoir with 34% emulsion phase reservoir. Combining the drug reservoir types improves the elution rate by maintaining the upfront burst of ropivacaine necessary for a robust peripheral nerve block and raising the elution rate later between 48 and 96 hours.

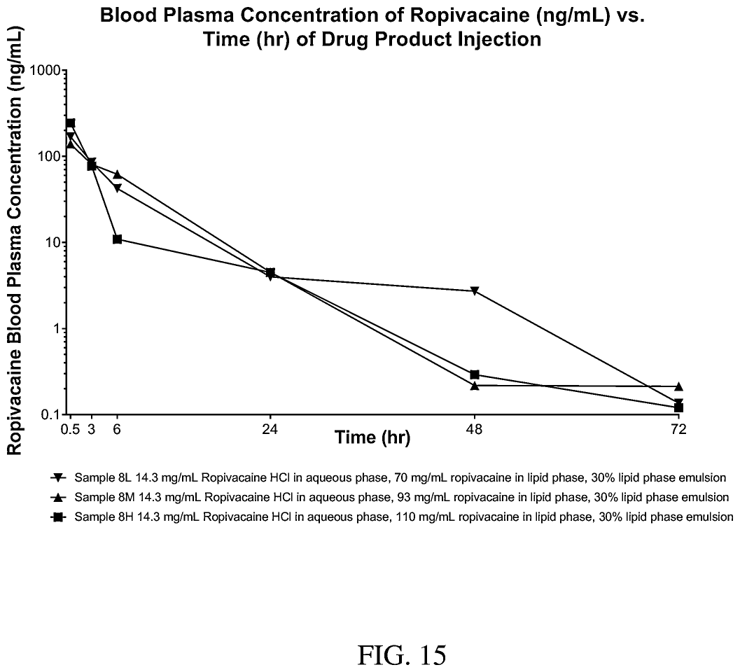

FIG. 15 shows how changing the ropivacaine loading in the emulsion phase does little to improve the elution rate at 72 hrs.

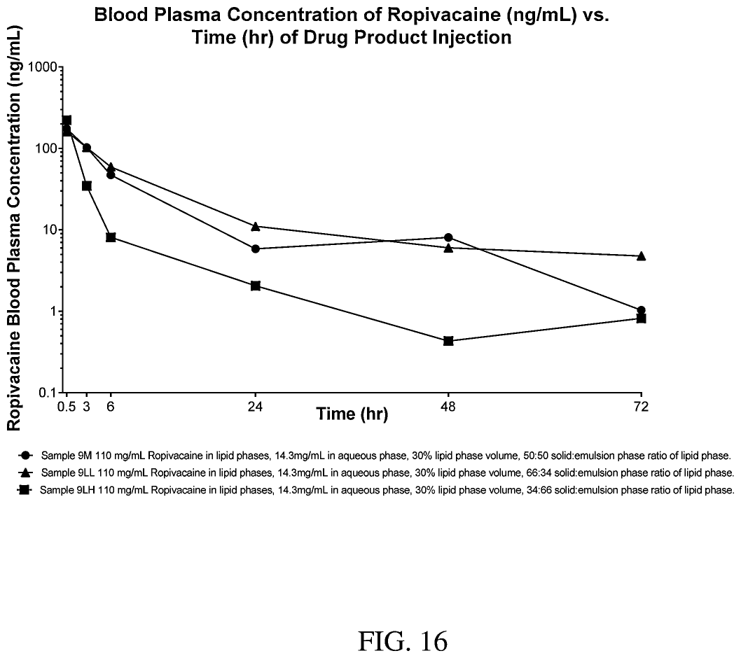

FIG. 16 compares various ratios of solid phase to emulsion phase drug reservoirs on elution rate. The best elution rate profile was shown by the 66:34 solid phase: emulsion phase formulation. This formulation generated a robust upfront dose that matches the Naropin control and then continues to deliver ropivacaine at an anesthetic dose past 72 hours. The 50:50 formulation may be more suitable for some surgical applications in which the anesthetic is more potent and a faster drop at 72 hours is desired.

### Example 9

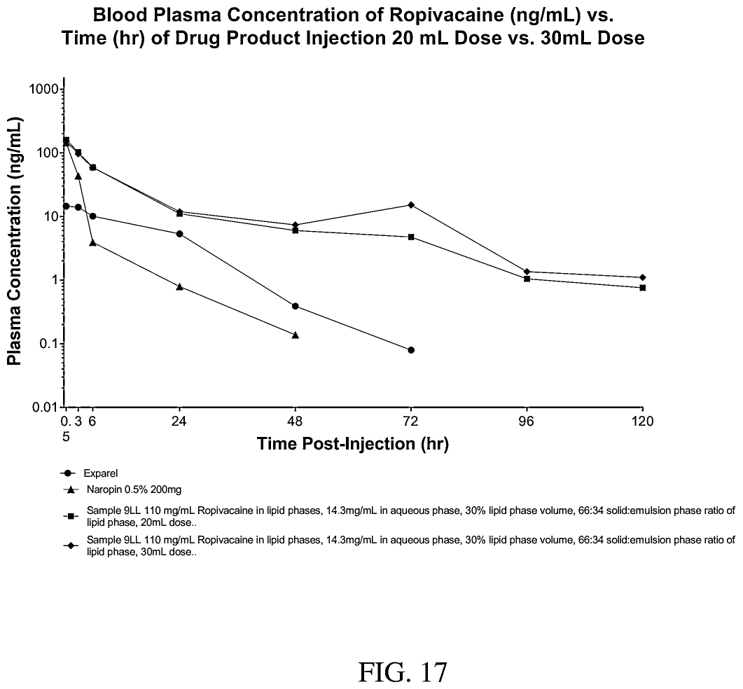

FIG. 17 shows the impact of increasing the dose of the sample 9LL formulation from 20 mL total dose delivered to 30 mL total dose delivered. The formulations performed similarly except for a peak around 72 hours which is due to the higher amount of MPs as a result of the increased dose.

### Example 10

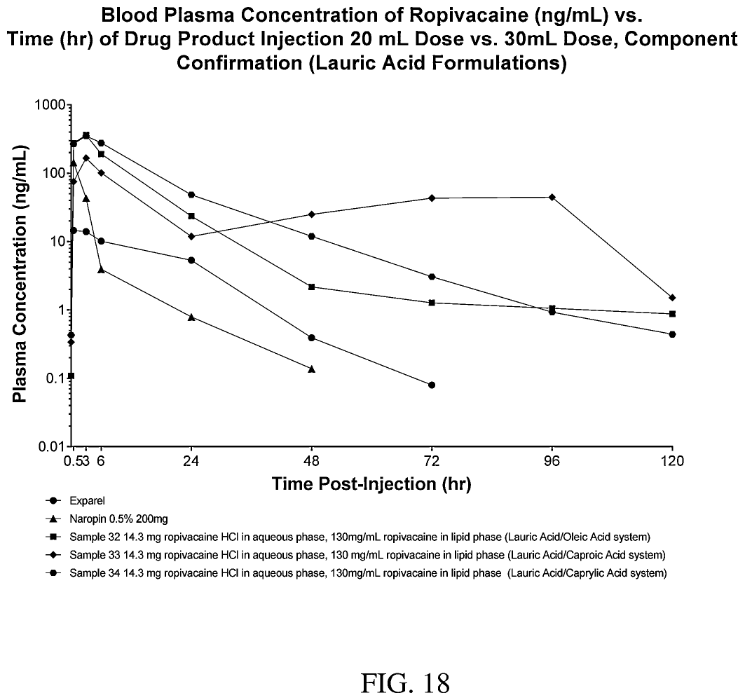

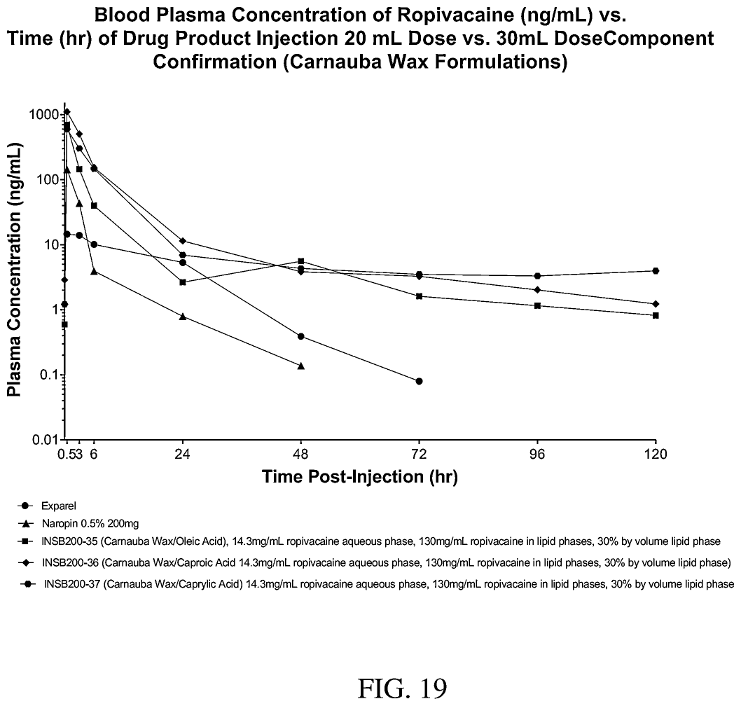

Previous formulations have been created using a stearic acid/oleic acid system, but it is possible to mimic this performance with other fatty acids. FIG. 18 shows a comparison of lauric acid, capric acid formulations to stearic acid and oleic acid formulations. FIG. 19 shows a comparison of carnauba wax/oleic acid formulations to controls. Fatty acid based drug product systems can use a plurality of fatty acids. Lauric acid can be combined with oleic, caproic, and caprylic acids to form similar performing formulations. Caproic acid was shown to be able to elute at a higher rate after 48 hrs.

### Example 12

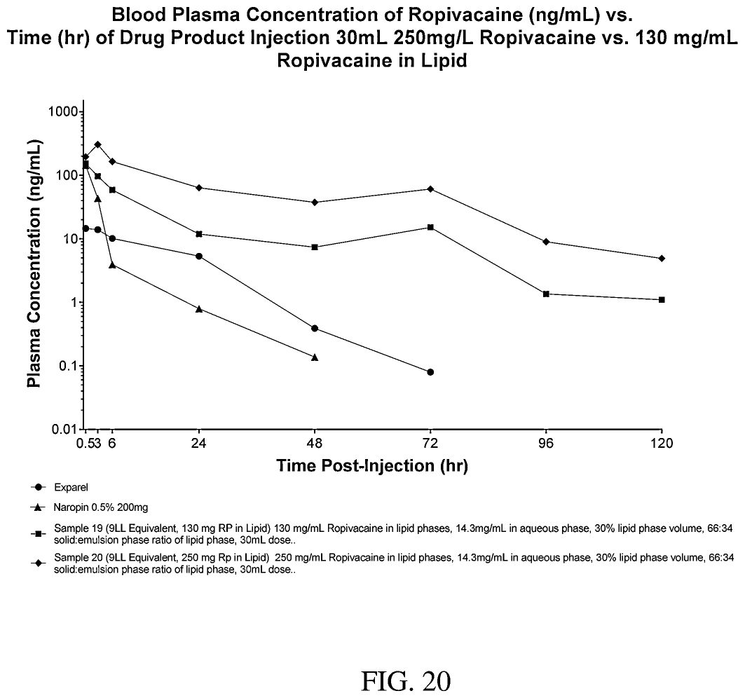

FIG. 20 shows a comparison between a solid phase fatty acid microparticle and a liquid (emulsion) phase drug reservoir. In this example both drug reservoir phases contain 90 mg/g ropivacaine in the reservoir phase. The solid phase does not release as much ropivacaine in the first 24 hrs and provides a stable release of ropivacaine beyond 24 hrs. It is envisioned that the two phases can be added together to create a new formulation that has an upfront burst with a higher elution rate beyond 24 hrs.

Although the disclosure has been described with reference to preferred embodiments, persons skilled in the art will recognize that changes may be made in form and detail without departing from the spirit and scope of the disclosed apparatus, systems and methods.

1. A composition for treating post-surgical pain comprising:
an aqueous carrier; and
a lipid phase comprising an anesthetic agent, the lipid phase dispersed within the aqueous carrier.

2. The composition of claim 1, wherein the aqueous carrier is hydrogel comprised of tyramine substituted hyaluronic acid, wherein the hydrogel is formed through di-tyramine crosslinking and wherein the degree of tyramine substitution of hyaluronic acid hydroxyl groups is about 0.5% to about 3%.

3. The composition of claim 1, wherein the lipid phase comprises a plurality of lipid microparticles.

4. The composition of claim 1, wherein the lipid phase is emulsified within the aqueous carrier.

5. The composition of claim 3, wherein a salt form of the anesthetic unbound by the plurality of lipid microparticles is dissolved in the aqueous carrier.

6. The composition of claim 5, wherein the volumetric ratio between the aqueous carrier and the lipid microparticles is from about 70-80 the aqueous carrier to about 30-20 lipid microparticles.

7. The composition of claim 3, wherein the lipid microparticles comprise one or more fatty acids having an even number of carbons.

8. The composition of claim 3, wherein the lipid microparticles comprise one or more fatty acids having an odd number of carbons.

9. The composition of claim 7, wherein the one or more fatty acids are chosen from: stearic acid, oleic acid, myristic acid, caprylic acid, capric acid, lauric acid, palmitic acid, arachidic acid, lignoceric acid, cerotic acid, and mixtures of the forgoing and wherein the melting point of the lipid microparticle is above 37° C.

10. The composition of claim 9, wherein the one or more fatty acids comprise a mixture of steric acid and oleic acid and wherein the ratio of steric acid to oleic acid is about 90:10.

11. The composition of claim 7, wherein in the lipid microparticles comprise about 12% myristic acid, about 32% palmitic acid, about 10% stearic acid, and about 10% oleic acid.

12. The composition of claim 9, wherein the lipid microparticles comprise a mixture of lauric acid and caprylic acid, caproic acid, and/or oleic acid.

13. The composition of claim 3, wherein the lipid microparticle comprises a paraffin, a triglyceride, and/or a wax.

14. The composition of claim 13, wherein the lipid microparticles comprise a mixture of carnauba wax and caprylic acid, caproic acid, and/or oleic acid.

15. The composition of claim 3, wherein the plurality of lipid microparticles comprises a first plurality of lipid microparticles and a second plurality of lipid microparticles and wherein the first plurality of lipid microparticles is solid at about 37° C. and the second plurality of lipid microparticles is liquid at 37° C.

16. The composition of claim 3, wherein the lipid microparticle is not a liposome.

17. The composition of claim 3, wherein the lipid microparticle ranges in size from about 1 μm to about 20 μm.

18. The composition of claim 17, wherein the lipid microparticle ranges in size from about 4 μm to about 8 μm.

19. The composition of claim 3, wherein the anesthetic agent comprises ropivacaine.

20. The composition of claim 19, wherein the ropivacaine is present in the lipid microparticles in an amount of from about 1 to about 25% by weight.

21. A composition for treating post-surgical pain comprising:
an aqueous carrier;
a first lipid phase comprising a plurality of lipid microparticles comprising an anesthetic agent and dispersed within the aqueous carrier; and
a second lipid phase comprising an anesthetic agent dissolved in one or more lipids and emulsified into to the aqueous phase.

22. The composition of claim 21, wherein a salt form of the anesthetic agent, not present in the first lipid phase or the second lipid phase, is dissolved in the aqueous carrier.

23. The composition of claim 22, wherein the one or more lipids of the second lipid phase is one or more fatty acids and wherein the second lipid phase is emulsified into the aqueous phase.

24. The composition of claim 23, wherein the one or more fatty acids of the second lipid phase are a mixture of stearic acid and oleic acid.

25. The composition of claim 21, wherein the volumetric ratio of the first lipid phase and the second lipid phase is about 66:34.

26. A method of treating post-operative pain in a subject in need thereof comprising administering to the subject and effective amount of a composition comprising:
an immiscible carrier phase; and
a plurality of lipid microparticles dispersed within the immiscible carrier phase comprising an anesthetic agent.

27. The method of claim 26, wherein the immiscible carrier phase is a hydrogel, a viscous liquid, a stable emulsion, or a cream.

28. The method of claim 27, wherein the immiscible carrier phase is a hydrogel.

29. The method of claim 28, wherein the hydrogel is comprised of tyramine substituted hyaluronic acid and wherein the anesthetic agent is ropivacaine.

30. The method of claim 29, wherein about 20 mL of the composition is administered to the subject and wherein the composition provides pain relief for about 72 hours.

31. The method of claim 29, wherein the composition is delivered near a never or nerve bundle of a subject and wherein the nerve or nerve bundle innervates the surgical incision area of the subject.
